(19) United States
(12) Patent Application Publication
(10) Pub. No.: US 2022/0054649 A1
(43) Pub. Date: Feb. 24, 2022

(54) ANTIBODY-DRUG CONJUGATES

(71) Applicant: SPIREA LIMITED, Harrogate (GB)

(72) Inventors: Myriam Ouberai, Harrogate (GB); Théophile Baïssas, Harrogate (GB)

(73) Assignee: SPIREA LIMITED, Harrogate (GB)

(21) Appl. No.: 17/415,624

(22) PCT Filed: Dec. 19, 2019
(86) PCT No.: PCT/GB2019/053629

§ 371 Date: Jun. 17, 2021

(30) Foreign Application Priority Data
Dec. 20, 2018 (GB) 1820864.5

(51) Int. Cl. A61K 47/68; A61K 47/60; A61K 38/07; A61P 35/00

(52) U.S. Cl. CPC A61K 47/6855; A61K 47/6889; A61P 35/00; A61K 38/07; A61K 47/60

(57) ABSTRACT

The present invention relates to antibody-drug conjugates comprising (i) an antibody or antigen-binding fragment thereof, (ii) a polymer comprising a particular repeat unit, which is covalently bound to one or more biologically active moieties, such as small molecule drugs, optionally via a linker, and (iii) a polymer-antibody linker moiety which is covalently bound to both the polymer and the antibody or antigen-binding fragment thereof. Additionally, the present invention relates to pharmaceutical compositions comprising the antibody-drug conjugates and to use of the antibody-drug conjugates in medicine.

### FIELD OF THE INVENTION

The present invention relates to antibody-drug conjugates comprising (i) an antibody or antigen-binding fragment thereof, (ii) a polymer comprising a particular repeat unit, which is covalently bound to one or more biologically active moieties, such as small molecule drugs, optionally via a linker, and (iii) a polymer-antibody linker moiety which is covalently bound to both the polymer and the antibody or antigen-binding fragment thereof. Additionally, the present invention relates to pharmaceutical compositions comprising the antibody-drug conjugates and to use of the antibody-drug conjugates in medicine.

### BACKGROUND INFORMATION

Antibody drug conjugates (ADCs) are a class of highly potent biopharmaceutical drugs, which have various therapeutic uses. For example, in the oncology field, ADCs can be used to target cancerous cells using an antibody on which a cytotoxic drug is attached via a linker. Despite these benefits, the development of ADCs has been limited due to the low drug-to-antibody ratios (DARs) of 3-4 that can be typically achieved. Often, with conventional ADCs, only one drug can be attached to the antibody per linker. This restriction limits the therapeutic index of ADCs and the range of drugs that can be used in ADCs, since only highly cytotoxic drugs can be employed. This also increases the prevalence of adverse reactions in patients. In addition, attempts to date to increase the DAR have resulted in aggregation of the ADC, rendering it ineffective.

There is therefore a need for new ADCs which can support a high DAR but which also have desirable physicochemical properties, such as high aqueous solubility and stability.

### SUMMARY OF THE INVENTION

The present invention provides an ADC containing a specific polymeric linker, which enables good stability and high solubility in aqueous solution. The specific polymeric linker used in the present invention can also support a high DAR, and is able to conjugate many different biologically active molecules (typically, 4 or more, 8 or more, preferably 12 or more, yet more preferably 16 or more, and most preferably up to 20 or more biologically active molecules) to a single antibody. Such a high DAR enables an improved therapeutic index.

Furthermore, the specific polymer used in the ADCs of the present invention may also enable the release rate of the biologically active molecules from the conjugate to be controlled. This release rate depends on the degradation of the covalent polymer-drug or linker-drug bonds within the ADC. Different types of covalent linkage will hydrolyse under different conditions of (e.g.) pH.

The specific polymer used in the ADCs of the present invention also enables multiple different types of drug moiety to be conjugated to the polymer. That can be useful, in particular, in achieving targeted combination therapy using two or more active agents. Combination therapies are particularly useful in oncology and the treatment of infectious diseases. The drugs used in combination therapies often have complimentary modes of action and/or have additive or synergistic therapeutic effects. The treatment protocols employing multiple drugs are, however, invariably complicated and intensive. Frequent drug dosing and concomitant administration of several different drugs at a given point in time is commonplace. Such complicated protocols tend to have lower patient compliance and tolerance than more straightforward protocols. The ability to conjugate multiple drugs to a single antibody with high DAR and favourable physicochemical properties therefore offers new opportunities in combination therapies.

The present invention accordingly provides an antibody-drug conjugate comprising:
(i) an antibody or antigen-binding fragment thereof,
(ii) a polymer comprising a repeat unit of Formula (I′):
   wherein:
   each n and each p is independently 0 or an integer between 1 and 6;
   each m is independently 0 or an integer between 1 and 4, and preferably at least one m is 1;
   V is

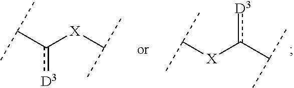

------- is a bond which may be absent or present;
each D¹ is independently O or L¹-B¹;
each D² is independently O or L²-B²
each D³ is independently O or L³-B³
L¹ is a linker group or a bond, L² is a linker group or a bond, L³ is a linker group or a bond, and each B¹, B² and B³ is a biologically active moiety;
provided that at least one D¹, D² or D³ group within the polymer is not O, and further provided that when D¹, D² or D³ is O, there is a double bond between the O atom and the carbon atom to which it is attached;
each q is an integer between 1 and 8;
X and Y are independently selected from O, NH, NR′ and S;
R′ is C₁₋₂₀ hydrocarbyl;
Q is selected from —CH₂(NMe(C═O)CH₂)ₒ—, -T¹O(CH₂CH₂O)ₛT²- and -T¹O(CH₂CH₂CH₂O)ₛT²-, wherein T¹ is selected from a divalent methylene, ethylene, propylene or butylene radical, and T² is selected from a divalent methylene, ethylene, propylene or butylene radical;
is an integer from 0 to 100; and
s is an integer from 0 to 150; and
(iii) a polymer-antibody linker which is covalently bonded to both the antibody and the polymer.

Preferably, the polymer comprises a repeat unit of Formula (I):

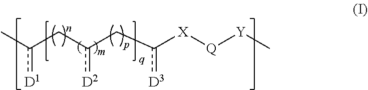

wherein the variables X, Y, D¹, D², D³, n, m and p are as set out above, and Q is selected from -T¹O(CH₂CH₂O)ₛT²- and -T¹O(CH₂CH₂CH₂O)ₛT²-.

Alternatively, the polymer comprises a repeat unit of Formula (I″):

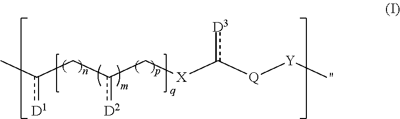

wherein the variables X, Y, Q, D¹, D², D³, n, m and p are as set out above. Preferably, in the repeat unit of Formula (I″), Q is CH₂(NMe(C═O)CH₂)ₒ—. More preferably, Q is CH—₂(NMe(C═O)CH₂)ₒ— and Y is —NMe.

In another aspect, the present invention also provides a pharmaceutical composition comprising an antibody-drug conjugate according to the invention, and a pharmaceutically acceptable excipient.

The present invention further provides an antibody-drug conjugate according to any the invention for use in the treatment of a disease or condition in a patient in need thereof.

The present invention further provides a method of treating a disease or condition as defined herein in a human patient, wherein said method comprises administration of at least one antibody-drug conjugate according to the invention to a patient in need thereof.

The present invention further provides the use of an antibody-drug conjugate according to the invention for the manufacture of a medicament for the treatment of a disease or condition as defined herein in a patient.

### BRIEF DESCRIPTION OF THE DRAWINGS

FIG. 1: GPC distribution plot monitoring of the synthesis of polyamide 1. The increase in the molecular weight of the polymers between the reaction mixture at 6 hours of stage 2 (line 1) and the treated polyamide 1 after 24 hours of stage 2 (line 2) is shown (GPC conditions: 2×(PLgel 10 μm MiniMIX-B 250×4.6 mm) columns, CHCl₃ 0.3 ml min-¹, PS standards).

FIG. 2: SEC distribution plot of polyamide 1. SEC conditions were as follows: PL-aquagel-OH 30, 8 μm, 300×7.5 mm, H₂O 0.5 ml min⁻¹, PEG standards. Area 1: Mₙ=13.9 kDa; Area 2: Mₙ=7.2 kDa; Area 3: Mₙ=3.7 kDa; Area 4: Mₙ=1.8 kDa; Area 5: Mₙ=1 kDa.

FIG. 3: Analytical RP-HPLC chromatogram of polyamide 1 using an Agilent ZORBAX 300SB-C18; 5 μm, 4.6×250 mm column with 1.3 mL min⁻¹ flow of H₂O (0.1% TFA) and AcCN as an eluent system and a gradient of 5% to 90% of AcCN in 15 minutes with UV-Vis detection at 215 nm.

FIG. 4: ¹H NMR of polyamide 1 in CDCl₃.

FIG. 5: Analytical RP-HPLC chromatogram monitoring of the maleimide functionalisation of polyamide 1 (Top panel at t=0 (polyamide 1), bottom panel after maleimide functionalisation (polyamide 2)) using an Agilent ZORBAX 300SB-C18; 5 μm, 4.6×250 mm column with 1.3 mL min⁻¹ flow of H₂O (0.1% TFA) and ACN as an eluent system and a gradient of 5% to 90% of AcCN in 15 minutes with UV-Vis detection at 215 nm.

FIG. 6: RP-HPLC semi-preparative chromatogram of polyamide 2 using an Agilent ZORBAX 300SB-C18; 5 m; 9.4×250 mm column with 4 mL min⁻¹ flow of H₂O (0.1% TFA) and AcCN as an eluent system and a gradient of 30% to 56% of AcCN in 13 minutes with UV-Vis detection at 215 nm. The vertical lines show the fraction collection regions.

FIG. 7: Analytical RP-HPLC chromatogram of polyamide 2 purified by semi-preparative RP-HPLC. Column used was Agilent ZORBAX 300SB-C18; 5 μm, 4.6×250 mm with 1.3 mL min⁻¹ flow of H₂O (0.1% TFA) and AcCN as an eluent system and a gradient of 5% to 90% of AcCN in 15 minutes with UV-Vis detection at 215 nm (top) and 250 nm (bottom).

FIG. 8: Analytical RP-HPLC chromatogram of polyamide 2 purified by semi-preparative RP-HPLC (line 2) compared to that of crude polymer (line 1) observed at 215 nm. Column used was Agilent ZORBAX 300SB-C18; 5 μm, 4.6×250 mm with 1.3 mL min⁻¹ flow of H₂O (0.1% TFA) and AcCN as an eluent system and a gradient of 5% to 90% of AcCN in 15 minutes.

FIG. 9: ¹H NMR of polyamide 2 after purification by RP-HPLC, in CDCl₃.

FIG. 10: Crude (left) and RP-HPLC purified (right) polyamide 2 at 50 mg mL-1 in water.

FIG. 11: LC-MS spectrum showing peak at m/z=598.95 for MMAE 5.

FIG. 12: ¹H NMR of polymer-MMAE conjugate 6 after purification by RP-HPLC, in CD₃CN.

FIG. 13: Cell viability assay on MMAE polyamide Trastuzumab ADC (Herceptin-MMAE-polymer conjugate)

### DETAILED DESCRIPTION OF THE INVENTION

### Definitions

As used herein, the term “polymer” refers to a compound comprising repeating units. Polymers usually have a polydispersity of greater than 1. Polymers generally comprise a backbone, side chains and termini. The backbone is the linear chain to which all side chains are pendant. The side chains are the groups that are pendant to the backbone or branch off the backbone. The termini are the ends of the backbone.

As used herein, the term “biologically active moiety” refers to any moiety that is derived from a biologically active molecule by abstraction of a hydrogen radical. A “biologically active molecule” is any molecule capable of inducing a biochemical response when administered in vivo. Typically, the biologically active molecule is capable of producing a local or systemic biochemical response when administered to an animal (or, preferably, a human); preferably the local or systemic response is a therapeutic activity. Preferred examples of biologically active molecules include drugs, peptides, proteins, peptide mimetics, antibodies, antigens, DNA, RNA, mRNA, small interfering RNA, small hairpin RNA, microRNA, PNA, foldamers, carbohydrates, carbohydrate derivatives, non-Lipinski molecules, synthetic peptides and synthetic oligonucleotides, and most preferably small molecule drugs.

As used herein, the term “small molecule drug” refers to a chemical compound which has known biological effect on an animal, such as a human. Typically, drugs are chemical compounds which are used to treat, prevent or diagnose a disease. Preferred small molecule drugs are biologically active in that they produce a local or systemic effect in animals, preferably mammals, more preferably humans. The small molecule drug may be referred to as a “drug molecule” or “drug”. Typically, the drug molecule has M_{W} less than or equal to about 5 kDa. Preferably, the drug molecule has M_{W} less than or equal to about 1.5 kDa. A more complete, although not exhaustive, listing of classes and specific drugs suitable for use in the present invention may be found in “Pharmaceutical Substances: Syntheses, Patents, Applications” by Axel Kleemann and Jurgen Engel, Thieme Medical Publishing, 1999 and the “Merck Index: An Encyclopedia of Chemicals, Drugs, and Biologicals”, edited by Susan Budavari et al., CRC Press, 1996, both of which are incorporated herein by reference in their entirety.

As used herein, the term “peptides” refers to biologically occurring or synthetic short chains of amino acid monomers linked by peptide (amide) bonds. The covalent chemical bonds are formed when the carboxyl group of one amino acid reacts with the amino group of another. The shortest peptides are dipeptides, consisting of 2 amino acids joined by a single peptide bond, followed by tripeptides, tetrapeptides, etc. A polypeptide is a long, continuous, and unbranched peptide chain. Hence, peptides fall under the broad chemical classes of biological oligomers and polymers, alongside nucleic acids, oligosaccharides and polysaccharides, etc.

As used herein, the term “amino acid” refers to any natural or synthetic amino acid, that is, an organic compound comprising carbon, hydrogen, oxygen and nitrogen atoms, and comprising both amino (—NH₂) and carboxylic acid (—COOH) functional groups. Typically, the amino acid is an α-, β-, γ- or δ-amino acid. Preferably, the amino acid is one of the twenty-two naturally occurring proteinogenic α-amino acids. Alternatively, the amino acid is a synthetic amino acid selected from α-Amino-n-butyric acid, Norvaline, Norleucine, Alloisoleucine, t-leucine, α-Amino-n-heptanoic acid, Pipecolic acid, α,β-diaminopropionic acid, α,γ-diaminobutyric acid, Ornithine, Allothreonine, Homocysteine, Homoserine, β-Alanine, β-Amino-n-butyric acid, β-Aminoisobutyric acid, γ-Aminobutyric acid, α-Aminoisobutyric acid, isovaline, Sarcosine, N-ethyl glycine, N-propyl glycine, N-isopropyl glycine, N-methyl alanine, N-ethyl alanine, N-methyl β-alanine, N-ethyl β-alanine, isoserine, α-hydroxy-γ-aminobutyric acid, Homonorleucine, O-methyl-homoserine, O-ethyl-homoserine, selenohomocysteine, selenomethionine, selenoethionine, Carboxyglutamic acid, Hydroxyproline, Hypusine, Pyroglutamic acid, aminoisobutyric acid, dehydroalanine, β-alanine, γ-Aminobutyric acid, δ-Aminolevulinic acid, 4-Aminobenzoic acid, citrulline, 2,3-diaminopropanoic acid and 3-aminopropanoic acid. An amino acid which possess a stereogenic centre may be present as a single enantiomer or as a mixture of enantiomers (e.g. a racemic mixture). Preferably, if the amino acid is an α-amino acid, the amino acid has L stereochemistry about the α-carbon stereogenic centre.

As used herein, the term “proteins” refers to biological molecules comprising polymers of amino acid monomers which are distinguished from peptides on the basis of size, and as an arbitrary benchmark can be understood to contain approximately 50 or more amino acids. Proteins consist of one or more polypeptides arranged in a biologically functional way, often bound to ligands such as coenzymes and cofactors, or to another protein or other macromolecule (DNA, RNA, etc.), or to complex macromolecular assemblies.

As used herein, the term “peptide mimetics” refers to small protein-like chains designed to mimic a peptide. They typically arise either from modification of an existing peptide, or by designing similar systems that mimic peptides, such as peptoids and β-peptides. Irrespective of the approach, the altered chemical structure is designed to advantageously adjust the molecular properties such as, stability or biological activity. This can have a role in the development of drug-like compounds from existing peptides. These modifications involve changes to the peptide that will not occur naturally (such as altered backbones and the incorporation of non-natural amino acids).

As used herein, the term “mRNA” refers to messenger RNA, a family of RNA molecules that convey genetic information from DNA to the ribosome, where they specify the amino acid sequence of the protein products of gene expression. Following transcription of primary transcript mRNA (known as pre-mRNA) by RNA polymerase, processed, mature mRNA is translated into a polymer of amino acids: a protein. As in DNA, mRNA genetic information is in the sequence of nucleotides, which are arranged into codons consisting of three bases each. Each codon encodes for a specific amino acid, except the stop codons, which terminate protein synthesis. This process of translation of codons into amino acids requires two other types of RNA: transfer RNA (tRNA), that mediates recognition of the codon and provides the corresponding amino acid, and ribosomal RNA (rRNA), that is the central component of the ribosome's protein-manufacturing machinery.

As used herein, the term “small interfering RNA” (siRNA) refers to a class of double-stranded RNA molecules, 20-25 base pairs in length. siRNA plays many roles, but it is most notable in the RNA interference (RNAi) pathway, where it interferes with the expression of specific genes with complementary nucleotide sequences. siRNA functions by causing mRNA to be broken down after transcription, resulting in no translation. siRNA also acts in RNAi-related pathways, e.g. as an antiviral mechanism or in shaping the chromatin structure of a genome.

As used herein, the term “small hairpin RNA” (shRNA) refers to an artificial RNA molecule with a tight hairpin turn that can be used to silence target gene expression via RNA interference (RNAi). Expression of shRNA in cells is typically accomplished by delivery of plasmids or through viral or bacterial vectors. shRNA is an advantageous mediator of RNAi in that it has a relatively low rate of degradation and turnover.

As used herein, the term “micro RNA” (miRNA) refers to a small non-coding RNA molecule (containing about 22 nucleotides) found in plants, animals, and some viruses, which functions in RNA silencing and post-transcriptional regulation of gene expression.

As used herein, the term “PNA” refers to peptide nucleic acid, an artificially synthesized polymer similar to DNA or RNA invented by Peter E. Nielsen (Univ. Copenhagen), Michael Egholm (Univ. Copenhagen), Rolf H. Berg (Risø National Lab), and Ole Buchardt (Univ. Copenhagen) in 1991. PNA's backbone is composed of repeating N-(2-aminoethyl)-glycine units linked by peptide bonds. The various purine and pyrimidine bases are linked to the backbone by a methylene bridge (—CH₂—) and a carbonyl group (—(C═O)—).

As used herein, the term “DNA” refers to deoxyribonucleic acid and derivatives thereof, the molecule that carries most of the genetic instructions used in the development, functioning and reproduction of all known living organisms and many viruses. Most DNA molecules consist of two biopolymer strands coiled around each other to form a double helix. The two DNA strands are known as polynucleotides since they are composed of simpler units called nucleotides. Each nucleotide is composed of a nitrogen-containing nucleobase—cytosine (C), guanine (G), adenine (A), or thymine (T)—as well as a monosaccharide sugar called deoxyribose and a phosphate group. The nucleotides are joined to one another in a chain by covalent bonds between the sugar of one nucleotide and the phosphate of the next, resulting in an alternating sugar-phosphate backbone. According to base pairing rules (A with T, and C with G), hydrogen bonds bind the nitrogenous bases of the two separate polynucleotide strands to make double-stranded DNA.

As used herein, the term “foldamer” refers to a discrete chain molecule or oligomer that folds into a conformationally ordered state in solution. They are artificial molecules that mimic the ability of proteins, nucleic acids, and polysaccharides to fold into well-defined conformations, such as helices and β-sheets. The structure of a foldamer is stabilized by non-covalent interactions between nonadjacent monomers.

As used herein, the term “carbohydrate” refers to biological molecule consisting of carbon (C), hydrogen (H) and oxygen (O) atoms, usually with a hydrogen:oxygen atom ratio of 2:1 (as in water); in other words, with the empirical formula Cₘ(H₂O)ₙ (where m could be different from n). Some exceptions exist; for example, deoxyribose, a sugar component of DNA, has the empirical formula C₅H₁₀O₄. Carbohydrates are technically hydrates of carbon; structurally it is more accurate to view them as polyhydroxy aldehydes and ketones. The term is most common in biochemistry, where it is a synonym of saccharide, a group that includes sugars, starch, and cellulose. The saccharides are divided into four chemical groups: monosaccharides, disaccharides, oligosaccharides, and polysaccharides.

As used herein, the term “non-Lipinski molecules” refers to molecules that do not conform to Lipinski's rule of five (also known as the Pfizer's rule of five or simply the Rule of five (RO5)), which is a rule of thumb to evaluate drug-likeness or to determine whether a chemical compound with a certain pharmacological or biological activity has properties that would make it a likely orally active drug in humans. The rule was formulated by Christopher A. Lipinski in 1997, based on the observation that most orally administered drugs are relatively small and moderately lipophilic molecules. The rule describes molecular properties important for a drug's pharmacokinetics in the human body, including their absorption, distribution, metabolism, and excretion (“ADME”). However, the rule does not predict if a compound is pharmacologically active.

As used herein, the term “acid-labile” refers to a bond which breaks in acidic conditions, e.g. a pH of <7.

As used herein, the term “direct bond” means that there are no intervening atoms. Thus, for example, a direct bond between a repeat unit and a drug means that a functional group of the drug is attached to an atom of the repeat unit, i.e. without the use of a linking group in-between.

As used herein, the term “C₁₋₂₀ hydrocarbyl” refers to any monovalent hydrocarbon radical comprising hydrogen and between 1 and 20 carbon atoms. Thus, hydrocarbyl groups consist of carbon and hydrogen. Examples of hydrocarbyl groups include alkyl, cycloalkyl, aryl, aralkyl, alkenyl, and alkynyl groups.

As used herein, the term “alkyl” refers to a linear or branched saturated monovalent hydrocarbon radical having the number of carbon atoms indicated in the prefix. Thus, the term “C₁₋₄ alkyl” refers to a linear saturated monovalent hydrocarbon radical of one to four carbon atoms or a branched saturated monovalent hydrocarbon radical of three or four carbon atoms, e.g. methyl, ethyl, n-propyl, iso-propyl, n-butyl, iso-butyl and tert-butyl.

Preferably, an alkyl group is a C₁₋₂₀ alkyl group, more preferably a C₁₋₁₂ alkyl group, yet more preferably a C₁₋₈ alkyl group, and most preferably a C₁₋₄ alkyl group.

As used herein, the term “alkylene” refers to a linear saturated divalent hydrocarbon radical or a branched saturated divalent hydrocarbon radical having the number of carbon atoms indicated in the prefix, e.g. methylene, ethylene, propylene, 1-methylpropylene, 2-methylpropylene, butylene, pentylene, and the like. Preferably, an alkylene group is a C₁₋₂₀ alkylene group, more preferably a C₁₋₁₂ alkylene group, yet more preferably a C₁₋₈ alkylene group, and most preferably a C₁₋₄ alkylene group.

As used herein, the term “alkenyl” refers to a linear or branched saturated monovalent hydrocarbon radical having the number of carbon atoms indicated in the prefix and containing at least one double bond. Thus, the term “C₂₋₆ alkenyl” refers to a linear saturated monovalent hydrocarbon radical of two to six carbon atoms having at least one double bond, or a branched saturated monovalent hydrocarbon radical of three to six carbon atoms having at least one double bond, e.g. ethenyl, propenyl, 1,3-butadienyl, (CH₂)₂CH═C(CH₃)₂, CH₂CH═CHCH(CH₃)₂, and the like. Preferably, an alkenyl group is a C₂₋₂₀ alkenyl group, more preferably a C₂₋₁₂ alkenyl group, yet more preferably a C₂₋₈ alkenyl group, and most preferably a C₂₋₄ alkenyl group.

As used herein, the term “alkenylene” refers to a linear saturated divalent hydrocarbon radical or a branched saturated divalent hydrocarbon radical having the number of carbon atoms indicated in the prefix and containing at least one double bond, e.g. ethenylene, propenylene, 1-methylpropenylene, 2-methylpropenylene, butenylene, pentenylene, and the like. Preferably, an alkenylene group is a C₂₋₂₀ alkenylene group, more preferably a C₂₋₁₂ alkenylene group, yet more preferably a C₂₋₈ alkenylene group, and most preferably a C₂₋₄ alkenylene group.

As used herein, the term “alkynyl” refers to a linear or branched saturated monovalent hydrocarbon radical having the number of carbon atoms indicated in the prefix and containing at least one triple bond. Thus, the term “C₂₋₆ alkynyl” refers to a linear saturated monovalent hydrocarbon radical of two to six carbon atoms having at least one triple bond, or a branched saturated monovalent hydrocarbon radical of four to six carbon atoms having at least one double bond, e.g. ethynyl, propynyl, and the like. Preferably, an alkynyl group is a C₂₋₂₀ alkynyl group, more preferably a C₂₋₁₂ alkynyl group, yet more preferably a C₂₋₈ alkynyl group, and most preferably a C₂₋₄ alkynyl group.

As used herein, the term “alkynylene” refers to a linear saturated divalent hydrocarbon radical or a branched saturated divalent hydrocarbon radical having the number of carbon atoms indicated in the prefix and containing at least one triple bond, e.g. ethynylene, propynylene, 1-methylpropynylene, 2-methylpropynylene, butynylene, pentynylene, and the like. Preferably, an alkynylene group is a C₂₋₂₀ alkynylene group, more preferably a C₂₋₁₂ alkynylene group, yet more preferably a C₂₋₈ alkynylene group, and most preferably a C₂₋₄ alkynylene group.

As used herein, the term “cycloalkyl” refers to a cyclic saturated monovalent hydrocarbon radical of three to ten carbon atoms, e.g. cyclopropyl, cyclobutyl, cyclopentyl, or cyclohexyl, and the like.

As used herein, the term “cycloalkylene” refers to a cyclic saturated divalent hydrocarbon radical of three to ten carbon atoms, e.g. cyclopropylene, cyclobutylene, cyclopentylene, or cyclohexylene, and the like. Preferably, a cycloalkylene group is a C₃₋₁₀ cycloalkylene group, more preferably a C₃₋₈ cycloalkylene group, and most preferably a C₃₋₆ cycloalkylene group.

As used herein, the term “heterocycyl” refers to a saturated or unsaturated monovalent monocyclic group of 4 to 8 ring atoms in which one or two ring atoms are heteroatoms selected from N, O, or S(O)ₙ, where n is an integer from 0 to 2, the remaining ring atoms being C. The heterocyclyl ring is optionally fused to a (one) aryl or heteroaryl ring as defined herein provided the aryl and heteroaryl rings are monocyclic. Additionally, one or two ring carbon atoms in the heterocyclyl ring can optionally be replaced by a —CO— group. More specifically the term heterocyclyl includes, but is not limited to, pyrrolidino, piperidino, homopiperidino, 2-oxopyrrolidinyl, 2-oxopiperidinyl, morpholino, piperazino, tetrahydropyranyl, thiomorpholino, and the like. When the heterocyclyl ring is unsaturated it can contain one or two ring double bonds, provided that the ring is not aromatic.

As used herein, the term “heterocyclylene” refers to a saturated or unsaturated divalent monocyclic group of 4 to 8 ring atoms in which one or two ring atoms are heteroatoms selected from N, O, or S(O)ₙ, where n is an integer from 0 to 2, the remaining ring atoms being C. The heterocyclylene ring is optionally fused to a (one) aryl or heteroaryl ring as defined herein provided the aryl and heteroaryl rings are monocyclic. Additionally, one or two ring carbon atoms in the heterocyclylene ring can optionally be replaced by a —CO— group. More specifically the term heterocyclylene includes, but is not limited to, pyrrolidinylene, piperidinylene, homopiperidinylene, 2-oxopyrrolidinylene, 2-oxopiperidinylene, morpholinylene, piperazinylene, tetrahydropyranylene, thiomorpholinylene, and the like. When the heterocyclylene ring is unsaturated it can contain one or two ring double bonds, provided that the ring is not aromatic.

As used herein, the term “aryl” refers to a monovalent monocyclic or bicyclic aromatic hydrocarbon radical of 6 to 10 ring atoms, e.g. phenyl or naphthyl, and the like.

As used herein, the term “arylene” refers to a divalent monocyclic or bicyclic aromatic hydrocarbon radical of 6 to 10 ring atoms, e.g. phenyl or naphthyl, and the like. Preferably, the arylene group is phenylene or naphthylene.

As used herein, the term “aralkyl” refers to an -(alkylene)-R radical where R is aryl as defined above. Preferably, the alkylene group is a C₁₋₂₀ alkylene group, more preferably a C₁₋₁₂ alkylene group, yet more preferably a C₁₋₈ alkylene group, and most preferably a C₁₋₄ alkylene group.

As used herein, the term “aralkylene” refers to an -(alkylene)-R divalent radical where R is arylene as defined above. Preferably, the aralkylene group is a C₇₋₂₀ aralkylene group, more preferably a C₇₋₁₄ aralkylene group, and most preferably a C₇₋₁₀ aralkylene group.

As used herein, the term “heteroaryl” refers to a monovalent monocyclic or bicyclic aromatic radical of 5 to 10 ring atoms where one or more, preferably one, two, or three, ring atoms are heteroatom selected from N, O, or S, the remaining ring atoms being carbon. Representative examples include, but are not limited to, pyrrolyl, thienyl, thiazolyl, imidazolyl, furanyl, indolyl, isoindolyl, oxazolyl, isoxazolyl, benzothiazolyl, benzoxazolyl, quinolinyl, isoquinolinyl, pyridinyl, pyrimidinyl, pyrazinyl, pyridazinyl, triazolyl, tetrazolyl, and the like.

As used herein, the term “heteroarylene” refers to a divalent monocyclic or bicyclic aromatic radical of 5 to 10 ring atoms where one or more, preferably one, two, or three, ring atoms are heteroatom selected from N, O, or S, the remaining ring atoms being carbon. Representative examples include, but are not limited to, pyrrolylene, thienylene, thiazolylene, imidazolylene, furanylene, indolylene, isoindolylene, oxazolylene, isoxazolylene, benzothiazolylene, benzoxazolylene, quinolinylene, isoquinolinylene, pyridinylene, pyrimidinylene, pyrazinylene, pyridazinylene, triazolylene, tetrazolylene, and the like.

As used herein, the term “heteroaralkyl” refers to an -(alkylene)-R radical where R is heteroaryl as defined above. Preferable alkylene groups are as defined for aralkyl groups above.

As used herein, the term “heteroaralkylene” refers to an -(alkylene)-R divalent radical where R is heteroarylene as defined above. Preferably, the heteroaralkylene group is a C₆₋₂₀ heteroaralkylene group, more preferably a C₆₋₁₄ heteroaralkylene group, and most preferably a C₆₋₁₀ heteroaralkylene group.

Optional substituents that may be present on alkyl, alkylene, alkenyl, alkenylene, alkylnyl, alkynylene, cycloalkyl, cycloalkylene, heterocyclyl, heterocyclylene, aryl, arylene, aralkyl, aralkylene, heteroaryl, heteroarylene, heteroaralkyl and heteroaralkylene groups include C₁₋₁₆ alkyl or C₁₋₁₆ cycloalkyl wherein one or more non-adjacent C atoms may be replaced with O, S, N, C═O and —COO—, substituted or unsubstituted C₅₋₁₄ aryl, substituted or unsubstituted C₅₋₁₄ heteroaryl, C₁₋₁₆ alkoxy, C₁₋₁₆ alkylthio, halo, cyano and aralkyl.

As used herein, the term “alkoxy” refers to an —OR radical where R is alkyl as defined above, e.g., methoxy, ethoxy, n-propoxy, iso-propoxy, n-butoxy, iso-butoxy, tert-butoxy and the like. Preferably, an alkoxy group is a C₁₋₂₀ alkoxy group, more preferably a C₁₋₁₂ alkoxy group, yet more preferably a C₁₋₈ alkoxy group, and most preferably a C₁₋₄ alkoxy group.

As used herein, the term “alkylthio” refers to an —SR radical where R is alkyl as defined above. Preferably, an alkylthio group is a C₁₋₂₀ alkylthio group, more preferably a C₁₋₁₂ alkylthio group, yet more preferably a C₁₋₈ alkylthio group, and most preferably a C₁₋₄ alkylthio group.

As used herein, the term “halo” refers to fluoro, chloro, bromo, or iodo, preferably fluoro or chloro.

As used herein, the term “keto group” refers to a carbonyl group, wherein the carbon atom of the carbonyl is also bonded to two carbon atoms.

As used herein, the term “hydrazine” refers to a group of the formula —NH—NH₂.

As used herein, the term “hydrazide” refers to a group of formulae R′(CO)—NH—NH₂ wherein R′ may be hydrogen or C₁₋₂₀ hydrocarbyl.

As used herein, the term “hydrazone” refers to a group of the formula ═N—NH—.

As used herein, the term “amine” refers to a group of the formula —NH₂, NHR or NR₂, wherein R is a C₁₋₂₀ hydrocarbyl group.

As used herein, the term “imine” refers to a group of the formula ═N—.

As used herein, the term “hydroxyl” refers to a group of the formula —OH.

As used herein, the term “ketal” refers to a group of the formula —C(OR)₂— wherein each R is C₁₋₂₀ hydrocarbyl or the two R groups together form a hydrocarbyl ring.

As used herein, the term “thiol” refers to a group of the formula —SH.

As used herein, the term “thioketal” refers to a group of the formula —C(SR)₂— wherein each R is C₁₋₂₀ hydrocarbyl or the two R groups together form a hydrocarbyl ring.

As used herein, the term “oxime” refers to a group of the formula ═N—O—.

As used herein, the term “aminoxy” or “hydroxylamine” refers to a group of the formula —O—NH₂. R—O—NH₂ refers to alkoxylamine.

As used herein, the term “Mₙ” as applied to a polymer refers to the number average molecular weight of the polymer.

As used herein, the term “M_{w}” as applied to a polymer refers to the weight average molecular weight of the polymer.

As used herein, the term “polydispersity” (also referred to as PD or Ð_{M}) refers to the ratio of the weight average molecular weight and the number average molecular weight of a polymer, i.e. Ð_{M}=M_{w}/Mₙ. It is a measure of the uniformity of a polymer sample. A low polydispersity indicates a narrow distribution of molecular mass within the polymer sample, and a high polydispersity indicates a broad distribution of molecular mass within the polymer sample.

### Antibody-Drug Conjugates

The present invention relates to an antibody-drug conjugate comprising (i) an antibody or antigen-binding fragment thereof, (ii) a polymer comprising a particular repeat unit, which is covalently bound to one or more biologically active moieties, such as small molecule drugs, optionally via a linker, and (iii) a polymer-antibody linker moiety which is covalently bound to both the polymer and the antibody or antigen-binding fragment thereof. Linker groups for attaching biologically active moieties to a polymer repeat unit are well-known in the art. Advantageously the biologically active moiety is not released from the polymer until the covalent bond between the polymer and the biologically active moiety or between the linker group and the biologically active moiety is broken, e.g. hydrolysed. The location of release of the biologically active moiety and the rate of release of the biologically active moiety can therefore be controlled by selecting an antibody that directs the ADC to the site of action, and tailoring the nature of the bond between the polymer and the biologically active moiety, or between the linker group and the biologically active moiety.

The antibody-drug conjugate of the invention comprises:
(i) an antibody or antigen-binding fragment thereof,
(ii) a polymer comprising a repeat unit of Formula (I′):

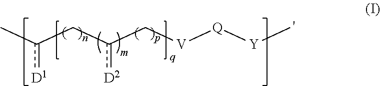

wherein:
each n and each p is independently 0 or an integer between 1 and 6;
each m is independently 0 or an integer between 1 and 4, and preferably at least one m is 1;
V is

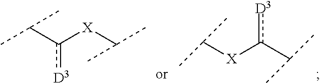

------- is a bond which may be absent or present;
each D¹ is independently O or L₁-B¹;
each D² is independently O or L²-B²
each D³ is independently O or L³-B³
wherein L¹ is a linker group or a bond, L² is a linker group or a bond, L³ is a linker group or a bond, and each B¹, B² and B³ is a biologically active moiety;
provided that at least one D¹, D² or D³ group within the polymer is not O, and further provided that when D¹, D² or D³ is O, there is a double bond between the O atom and the carbon atom to which it is attached;
each q is an integer between 1 and 8;
X and Y are independently selected from O, NH, NR′ and S;
R′ is C₁₋₂₀ hydrocarbyl;
Q is selected from —CH₂(NMe(C═O)CH₂)ₒ—, -T¹O(CH₂CH₂O)ₛT²- and -T¹O(CH₂CH₂CH₂O)ₛT²-, wherein T¹ is selected from a divalent methylene, ethylene, propylene or butylene radical, and T² is selected from a divalent methylene, ethylene, propylene or butylene radical;
is an integer from 0 to 100; and
s is an integer from 0 to 150; and
(iii) a polymer-antibody linker which is covalently bonded to both the antibody and the polymer.

Preferably, the polymer comprises a repeat unit of Formula (I):

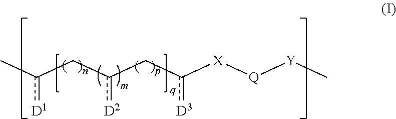

wherein the variables X, Y, D¹, D², D³, n, m and p are as set out above, an is selected from -T¹O(CH₂CH₂O)ₛT²- and -T¹O(CH₂CH₂CH₂O)ₛT²-.

Alternatively, the polymer comprises a repeat unit of Formula (I″):

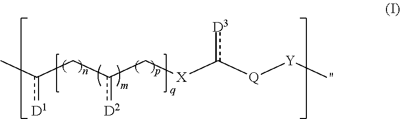

wherein the variables X, Y, Q, D¹, D², D³, n, m and p are as set out above. Preferably, in the repeat unit of Formula (I″), Q is CH₂(NMe(C═O)CH₂)ₒ—. More preferably, Q is CH—₂(NMe(C═O)CH₂)ₒ— and Y is —NMe.

Structural Features of the Antibody

This section sets out the possible structural features of an antibody present in the antibody-drug conjugates of the invention.

The term “antibody” as referred to herein includes whole antibodies and any antigen-binding fragment (i.e., “antigen-binding portion”) or single chains thereof, as well as variants thereof. An antibody may also be referred to as an immunoglobulin (Ig). An antibody refers to a glycoprotein comprising at least two heavy (H) chains and two light (L) chains inter-connected by disulfide bonds, or an antigen binding portion thereof. Each heavy chain is comprised of a heavy chain variable region (abbreviated herein as VH) and a heavy chain constant region. Each light chain is comprised of a light chain variable region (abbreviated herein as VL) and a light chain constant region. The variable regions of the heavy and light chains contain a binding domain that interacts with an antigen. An antigen is any agent that causes the immune system of an animal body to produce an immune response, e.g. chemicals, bacteria, viruses or pollen. The VH and VL regions can be further subdivided into regions of hypervariability, termed complementarity determining regions (CDR), interspersed with regions that are more conserved, termed framework regions (FR). The constant regions of the antibodies may mediate the binding of the immunoglobulin to host tissues or factors, including various cells of the immune system (e.g., effector cells) and the first component (Clq) of the classical complement system.

The antibody may be a monoclonal antibody or a polyclonal antibody. Typically, the antibody is a monoclonal antibody. Alternatively, the antibody is a polyclonal antibody. Polyclonal antibodies are antibodies that are derived from different B cell lines. A polyclonal antibody may comprise a mixture of different immunoglobulin molecules that are directed against a specific antigen. The polyclonal antibody may comprise a mixture of different immunoglobulin molecules that bind to one or more different epitopes within an antigen molecule. Polyclonal antibodies may be produced by routine methods such as immunisation with the antigen of interest. For example a mouse or sheep capable of expressing antibodies may be immunised with an immunogenic conjugate. The animals may optionally be capable of expressing human antibody sequences. Blood may be subsequently removed and the Ig fraction purified to extract the polyclonal antibodies.

Monoclonal antibodies (mAbs) are immunoglobulin molecules that are identical to each other and have a single binding specificity and affinity for a particular epitope. mAbs useful in the antibody-drug conjugates of the present invention can be produced by a variety of techniques, including conventional monoclonal antibody methodology, for example those disclosed in “Monoclonal Antibodies; A manual of techniques”, H Zola (CRC Press, 1988) and in “Monoclonal Hybridoma Antibodies: Techniques and Application”, SGR Hurrell (CRC Press, 1982).

The term “antigen-binding portion” of an antibody refers to a fragment of an antibody that retains the ability to specifically bind to an antigen, such as a protein, polypeptide or peptide. It has been shown that the antigen-binding function of an antibody can be performed by fragments of a full-length antibody. Examples of binding fragments encompassed within the term “antigen-binding portion” of an antibody include a Fab fragment, a F(ab′)₂ fragment, a Fab′ fragment, a Fd fragment, a Fv fragment, a dAb fragment and an isolated complementarity determining region (CDR). Single chain antibodies such as scFv and heavy chain antibodies such as VHH and camel antibodies are also intended to be encompassed within the term “antigen-binding portion” of an antibody.

These antibody fragments may be obtained using conventional techniques known to those of skill in the art, and the fragments may be screened for utility in the same manner as intact antibodies.

Antibody “fragments” as defined herein may be made by truncation, e.g. by removal of one or more amino acids from its N and/or C-terminal ends. Up to 10, up to 20, up to 30, up to 40 or more amino acids may be removed from the N and/or C terminal in this way. Fragments may also be generated by one or more internal deletions. A fragment may comprise of at least 10, at least 15, at least 20, at least 25, at least 30, at least 35, at least 40, at least 45, at least 55, at least 60, at least 65, at least 70, at least 75, at least 80, at least 85, at least 90, at least 95, at least 100, at least 105, at least 120, at least 150, at least 200, at least 250, at least 300 or at least 400 consecutive amino acids from an antibody or antibody variant sequence.

Preferably, the antibody in the antibody-drug conjugate of the present invention is selected from Gemtuzumab hP67.6 humanized IgG4, Brentuximab Chimeric IgG1, Trastuzumab Humanized IgG1, Inotuzumab G5/44 Humanized IgG4, Glembatumumab Fully human IgG1, Anetumab Anti-mesothelin fully humana IgG1, Mirvetuximabb M9346A Humanized IgG1, Depatuxizumabb (ABT-806) Humanized IgG1, Rovalpituzumab (SC16) Humanized IgG1, and Vadastuximabb Humanized IgG1.

Structural Features of the Polymer

This section sets out the possible structural features of the polymer present in the antibody-drug conjugates of the invention.

The polymer of the antibody-drug conjugates of the present invention is either derived from:

(i) a compound of Formula (IIa):

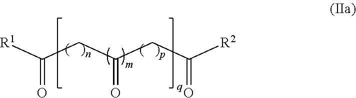

wherein R¹ and R² are each independently a leaving group, and n, m, p, q and R′ are as defined above for the repeat unit of Formula (I′), (I) or (I″);

(ii) a compound of Formula (IIb):

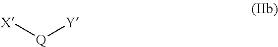

wherein X′ and Y′ are independently selected from OH, OR′, SH, SR′, NH₂, NHR′ and NR′₂, and Q and R′ are as defined above for the repeat unit of Formula (I′), (I) or (I″);

(iii) one or more biologically active molecules; and

(iv) optionally, one or more compounds selected from HL¹-LG, HL²-LG or HL³-LG,

wherein L¹ is a linker group, L² is a linker group, L³ is a linker group, and LG is a leaving group under addition-elimination reaction conditions; or is derived from:

(i) a compound of Formula (IIa′):

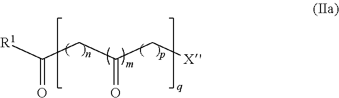

wherein R¹ is a leaving group, X′ is selected from OH, OR′, SH, SR′, NH₂, NHR′ and NR′₂, and n, m, p, q and R′ are as defined above for the repeat unit of Formula (I′), (I) or (I″);

(ii) a compound of Formula (IIb′):

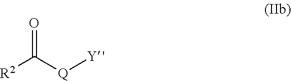

wherein R² is a leaving group, Y′ is selected from OH, OR′, SH, SR′, NH₂, NHR′ and NR′₂, and Q and R′ are as defined above for the repeat unit of Formula (I′), (I) or (I″);

(iii) one or more biologically active molecules; and

(iv) optionally, one or more compounds selected from HL¹-LG, HL²-LG or HL³-LG, wherein L¹ is a linker group, L² is a linker group, L³ is a linker group, and LG is a leaving group under addition-elimination reaction conditions.

In the polymer of the antibody-drug conjugates of the present invention, q may be 1, 2, 3, 4, 5, 6, 7 or 8. Preferably, however, q is 1, 2 or 3, still more preferably 1 or 2 and particularly preferably 1. When q is an integer greater than 1, each n, m and p present in the repeat unit of Formula (I′), (I) or (I″) may be the same or different.

Preferably, q is 1. Preferably therefore the polymer of the antibody-drug conjugates of the present invention is derived from a compound of Formula (IIc) or Formula (IIc′):

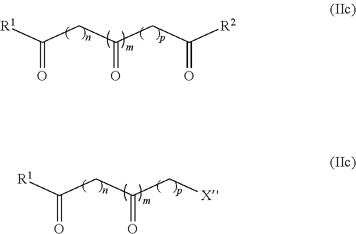

wherein R¹, R², R′, X′, n, m and p are as defined above in relation to Formula (IIa) and Formula (IIa′).

In the polymer of the antibody-drug conjugates of the present invention, each m may be 0, 1, 2, 3 or 4, provided that at least one m is 1, 2, 3 or 4. Preferably at least one m is 1. This ensures that at least one keto group is present in the repeat unit of Formula (I′), (I) or (I″). Preferably each m is 1 or 2 and still more preferably each m is 1. When m is 1, and both n and p are 1 or greater, the keto groups are spaced apart by at least one carbon atom and it is believed that this avoids steric clashes between biologically active moieties attached to the polymer.

In the polymer of the antibody-drug conjugates of the present invention, each n is 0, 1, 2, 3, 4, 5 or 6. Preferably each n is 1, 2 or 3, and still more preferably 1 or 2. In the polymer of the antibody-drug conjugates of the present invention, each p is 0, 1, 2, 3, 4, 5 or 6. Preferably each p is 0, 1 or 2, and still more preferably 0 or 1. Even more preferably, each p is 0. The n and p groups space apart the keto groups and advantageously enables a relatively high amount of biologically active molecule to be covalently bound to the polymer.

The polymers are preferably derived from a compound of Formula (IIa), (IIa′), (IIc) or (IIc′) wherein R¹ is selected from Cl, OH, OR′, SH, SR′, NH₂, NHR′, NR′₂, O-2-Cl-Trt, ODmb, O-2-PhⁱPr, O-EDOTn-Ph, OFm, ODmab and OCam. Still more preferably R¹ is selected from OMe, OEt, O^{t}Bu, O-2-Cl-Trt, ODmb, O-2-PhⁱPr, O-EDOTn-Ph, OFm, ODmab and OCam. Further preferred polymers are derived from a compound of Formula (IIa) or (IIc), or a compound of Formula (IIb′), wherein R² is selected from Cl, OH, OR′, SH, SR′, NH₂, NHR′, NR′₂, O-2-Cl-Trt, ODmb, O-2-PhⁱPr, O-aryl-EDOTn, OFm, ODmab and OCam. Still more preferably R² is selected from OMe, OEt, O^{t}Bu, O-2-Cl-Trt, ODmb, O-2-PhⁱPr, O-EDOTn-Ph, OFm, ODmab and OCam. R¹ and R² may be the same or different, but are preferably the same. Yet more preferably R¹ and R² are both selected from OMe, OEt, O^{t}Bu, O-2-Cl-Trt, ODmb, O-2-Ph¹Pr, O-aryl-EDOTn, OFm, ODmab and OCam. An example of a compound of Formula (IIa′) is amino-2-keto butyric acid.

As defined herein, 2-Cl-Trt refers to 2-chlorotrityl. As defined herein, Dmb refers to 2,4-dimethoxybenzyl. As defined herein, 2-Ph¹Pr refers to 2-phenylisopropyl. As defined herein, Fm refers to 9-fluorenylmethyl. As defined herein, Dmab refers to 4-(N-[1-(4,4-dimehtyl-2,6-dioxocyclohexylidene)-3-methylbutyl]-amino)benzyl. As defined herein, Cam refers to carbamoylmethyl. As defined herein, aryl-EDOTn refers to a moiety having the following formula:

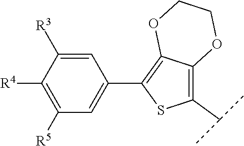

wherein R³ is H or OMe, R⁴ is H or OMe and R⁵ is H or OMe. Preferably, R³, R⁴ and R⁵ are selected such that (a) all of R³, R⁴ and R⁵ are H, (b) all of R³, R⁴ and R⁵ are OMe, (c) R³ and R⁴ are OMe and R⁵ is H, or (d) R³ and R⁴ are H and R⁵ is OMe.

When R¹ and/or R² comprise a R′ group, R′ is preferably a C₁₋₂₀ alkyl, more preferably a C₁₋₁₂ alkyl, yet more preferably a C₁₋₈ alkyl and especially preferably a C₁₋₄ alkyl. Representative examples of suitable alkyl groups include methyl, ethyl, propyl, isopropyl, butyl, isobutyl and tert-butyl. Methyl, ethyl and tert-butyl are particularly preferred alkyl groups.

The polymers are preferably derived from a compound of Formula (IIb) or (IIb′) wherein T¹ is —CH₂—, —CH₂CH₂—, —CH₂CH₂CH₂— or —CH₂CH₂CH₂CH₂—, more preferably wherein T¹ is —CH₂CH₂— or —CH₂CH₂CH₂—. The polymers are preferably derived from a compound of Formula (IIb) or (IIb′) wherein T² is —CH₂—, —CH₂CH₂—, —CH₂CH₂CH₂— or —CH₂CH₂CH₂CH₂—, more preferably wherein T² is —CH₂CH₂— or —CH₂CH₂CH₂—. T¹ and T² may be the same or different. Preferably, T¹ and T² are the same. Typically, the polymers are derived from a compound of Formula (IIb) in which both T¹ and T² are selected from —CH₂—, —CH₂CH₂—, —CH₂CH₂CH₂— and —CH₂CH₂CH₂CH₂—, preferably wherein both T¹ and T² are selected from —CH₂CH₂— and —CH₂CH₂CH₂—, and more preferably wherein both T¹ and T² are —CH₂CH₂CH₂—. Alternatively, the polymers may be derived from a compound of Formula (IIb) or (IIb′) wherein Q is CH₂(NMe(C═O)CH₂)ₒ—.

The polymers are further preferably derived from a compound of Formula (IIb) wherein X′ is OH, OR′, NH₂, or NHR′. Still more preferably X′ is OH or NH₂. Further preferred polymers are derived from a compound of Formula (IIb) or (IIb′) wherein Y′ is OH, OR′, NH₂, or NHR′. Still more preferably Y′ is OH or NH₂. X′ and Y′ may be the same or different, but are preferably the same. Yet more preferably X′ and Y′ are both OH. Thus, in a preferable embodiment, the compound of Formula (IIb) is a polyethyleneglycol (PEG) or a polypropylene glycol. Preferably in this case, the compound of Formula (IIb) is selected from PEG 400, PEG 500, PEG 600, PEG 1000, PEG 1500, PEG 2000, PEG 3000, PEG 4000 and PEG 5000. Yet more preferably, X′ and Y′ are both NH₂. Still more preferably, X′ and Y′ are both NH₂ and both T¹ and T² are —CH₂CH₂CH₂—. In this case the compound of Formula (IIb) is a poly(ethylene glycol) bis(3-aminopropyl) or a poly(propylene glycol) bis(3-aminopropyl). Most preferably, X′ and Y′ are both NH₂ and Q is —CH₂CH₂CH₂O(CH₂CH₂O)ₛCH₂CH₂CH₂—. In this case the compound of Formula (IIb) is a poly(ethylene glycol) bis(3-aminopropyl). Preferably the (poly(ethylene glycol) bis(3-aminopropyl) has a molecular weight of from 1000 to 2000, and more preferably has a molecular weight of 1500, i.e. is (poly(ethylene glycol) bis(3-aminopropyl) terminated) 1500. In another preferred embodiment, the polymers are derived from a compound of Formula (IIb′) wherein Q is CH₂(NMe(C═O)CH₂)ₒ— and Y′ is —NH₂ or —NHR′, preferably —NHR′, more preferably —NHMe. Yet more preferably, Q is —CH₂(NMe(C═O)CH₂)ₒ—, Y′ is —NHMe, and R² is OH or OR′. In this case, the compound of Formula (IIb′) is a poly(sarcosine) or an ester thereof. Preferably the poly(sarcosine) has a molecular weight of from 350 to 1800.

o is preferably an integer from 0 to 100, more preferably from 1 to 75, still more preferably from 2 to 50, and most preferably from 5 to 25.

s is preferably an integer from 0 to 150, more preferably from 1 to 100, still more preferably from 1 to 50, yet more preferably from 1 to 35, even more preferably from 3 to 30 and most preferably from 5 to 21.

Preferably, the polymer is derived from dimethyl-2-oxo-glutarate or dimethyl-3-oxo-glutarate. Further preferred polymers are derived from poly(ethylene glycol) or poly(ethylene glycol) bis(3-aminopropyl). Most preferably, the polymer is derived from dimethyl-2-oxo-glutarate and poly(ethylene glycol), dimethyl-2-oxo-glutarate and poly(ethylene glycol) bis(3-aminopropyl), dimethyl-3-oxo-glutarate and poly(ethylene glycol), or dimethyl-3-oxo-glutarate and poly(ethylene glycol) bis(3-aminopropyl).

In the repeat unit of Formula (I′), (I) or (I″), the dashed bonds may be present or absent. When it is absent there is a single bond to the moieties D¹, D² and/or D³. When it is present there is a double bond to the to the moieties D¹, D² and/or D³, or two single bonds to different atoms within the moieties D¹, D² and/or D³. When D¹, D² or D³ is an oxygen atom, there is a double bond between the oxygen atom and the carbon atom to which it is attached. Preferably when D¹ is L¹-B¹, D² is L²-B² or D³ is L³-B³, the dashed bond is present. Alternatively when D¹ is L¹-B¹, D² is L²-B² or D³ is L³-B³, the dashed bond is absent.

Preferably, the repeat unit of Formula (I′), (I) or (I″) is a repeat unit of Formula (I-i′), e.g. a repeat unit of Formula (I-i) or a repeat unit of Formula (I-I″), wherein q in Formula (I′), (I) or (I″) is 1:

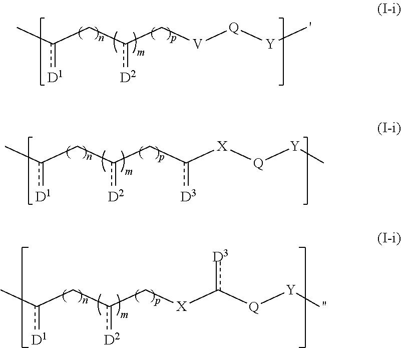

wherein n, m, p, V, D¹, D², D³, X, Y, and Q are as defined above in relation to Formula (I′), Formula (I) or Formula (I″).

In the polymer repeat unit of Formula (I′), (I), (I″) (I-i′), (I-i) or (I-i″), each D¹ is independently O or L¹-B¹, each D² is independently O or L²-B², and each D³ is independently O or L³-B³, wherein each L¹, L² and L³ is a linker group or a bond, and each B¹, B² and B³ is a biologically active moiety (e.g. a drug). A biologically active moiety is a moiety derived from a biologically active molecule (e.g. a drug) once that molecule has formed a covalent bond to either the backbone of the polymer repeat unit or, if present, a linker group. When the bond between the polymer repeat unit or the linker group and B¹, B² or B³ is broken, a compound H—B¹, H—B² or H—B³ is released, which is a biologically active molecule. Thus, as used herein, a “biologically active molecule” is a said biologically active moiety which is attached to a hydrogen atom rather than to the polymer repeat unit or linker group.

Typically, each L¹, L² and L³ is a bond. Thus, typically L¹ is a bond. Typically L² is a bond. Typically L³ is a bond. If L¹, L² or L³ is a bond, this means that D¹, D² or D³, respectively, is B¹, B² or B³, i.e. a biologically active moiety. In this case, said biologically active moiety is typically obtainable by reaction of a biologically active molecule H—B¹, H—B² or H—B³ with a carbonyl group, preferably a keto group, in the compound of Formula (IIa), (IIa′), (IIc) or (IIc′) from which the polymer repeat unit is derived. Typically, each B¹, B² and B³ is a moiety obtainable by a condensation between a nucleophile within a drug molecule and an electrophilic carbonyl carbon atom in the compound of Formula (IIa), (IIa′), (IIc) or (IIc′) from which the polymer repeat unit is derived.

Preferably, however, each L¹, L² and L³ is a linker group. In other words, typically the antibody-drug conjugates of the present invention comprise a linker between the polymer backbone and the biologically active moiety. Thus, preferably, L¹ is a linker group. Preferably, L² is a linker group. Preferably, L³ is a linker group. If L¹, L² or L³ is a linker group, this linker group may be any linker group suitable for connecting a biologically active moiety to the polymer backbone via covalent linkages. Such linker groups are well-known in the art. Preferably, L¹ has a molecular weight of from 14 to 4000 Da, more preferably from 28 to 2000 Da, still more preferably from 50 to 1000 Da, and yet more preferably from 100 to 500 Da. Preferably, L² has a molecular weight of from 14 to 4000 Da, more preferably from 28 to 2000 Da, still more preferably from 50 to 1000 Da, and yet more preferably from 100 to 500 Da. Preferably, L³ has a molecular weight of from 14 to 4000 Da, more preferably from 28 to 2000 Da, still more preferably from 50 to 1000 Da, and yet more preferably from 100 to 500 Da.

Preferable linker groups are selected from ═N—NH—Z¹-L′-Z²—, ═N—O—Z¹-L′-Z²—, ═N—Z¹-L′-Z²—, —NH—NH—Z¹-L′-Z²—, —NH—O—Z¹-L′-Z²—, —NH—Z¹-L′-Z²—, —O—Z¹-L′-Z²—S—Z¹-L′-Z²—,

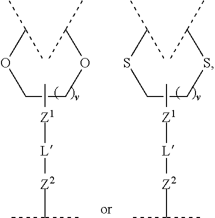

wherein.

L′ is selected from a bond, C₁₋₂₀ alkylene, C₁₋₂₀ alkenylene, C₁₋₂₀ alkynylene, C₆₋₁₀ arylene (e.g. phenylene or naphthylene), C₇₋₂₀ aralkylene, C₃₋₁₀ cycloalkylene, C₄₋₈ heterocycloalkylene, C₅₋₁₀ heteroarylene, C₆₋₂₀ heteroaralkylene, —(O—K)ᵢ—, —(NH—K)ᵢ—, —(NR′—K)ᵢ—, a polyester having a molecular weight of from 116 to 2000 Da, a polyamide having a molecular weight of from 114 to 2000 Da, and a moiety —W— wherein H—W—OH is an amino acid or a peptide containing from two to twenty naturally-occurring or synthetic amino acid subunits;

Z¹ is selected from —Z—(C═O)—, —Z—O(C═O)—, —Z—NH(C═O)—, —Z—NR′(C═O)—, —Z—S(C═O)—, —Z—(C═NH)—, —Z—O(C═NH)—, —Z—NH(C═NH)—, —Z—NR′(C═NH)—, —Z—S(C═NH)— and —Z—(C═NR′)—, —K—(O—K)ᵢ—, —K—(NH—K)ᵢ—, —K—(NR′—K)ᵢ—, —K(C═O)—(O—K—(C═O))ᵢ—, —K(C═O)—(NH—K—(C═O))ᵢ—, —K(C═O)—(NR′—K—(C═O))ᵢ—, and a moiety —P— wherein H₂N—P—OH is an amino acid or a peptide containing from two to twenty naturally-occurring or synthetic amino acid subunits; Z² is selected from a bond, —OZ—, —NHZ—, —NR′Z—, —SZ—, —S—, —ZS—, —OZS—, —NHZS—, —NR′ZS—, —SZS—, —Z—(C═O)—, —Z—O(C═O)—, —Z—NH(C═O)—, —Z—NR′(C═O)—, —Z—S(C═O)—, —Z—(C═NH)—, —Z—O(C═NH)—, —Z—NH(C═NH)—, —Z—NR′(C═NH)—, —Z—S(C═NH)—, —Z—(C═NR′)—, —Z—O(C═NR′)—, —Z—NH(C═NR′)—, —Z—NR′(C═NR′)—, —Z—S(C═NR′)—, —OZ—(C═O)—, —OZ—O(C═O)—, —OZ—NH(C═O)—, —OZ—NR′(C═O)—, —OZ—S(C═O)—, —OZ—(C═NH)—, —OZ—O(C═NH)—, —OZ—NH(C═NH)—, —OZ—NR′(C═NH)—, —OZ—S(C═NH)—, —OZ—(C═NR′)—, —OZ—O(C═NR′)—, —OZ—NH(C═NR′)—, —OZ—NR′(C═NR′)—, —OZ—S(C═NR′)—, —NHZ—(C═O)—, —NHZ—O(C═O)—, —NHZ—NH(C═O)—, —NHZ—NR′(C═O)—, —NHZ—S(C═O)—, —NHZ—(C═NH)—, —NHZ—O(C═NH)—, —NHZ—NH(C═NH)—, —NHZ—NR′(C═NH)—, —NHZ—S(C═NH)—, —NHZ—(C═NR′)—, —NHZ—O(C═NR′)—, —NHZ—NH(C═NR′)—, —NHZ—NR′(C═NR′)—, —NHZ—S(C═NR′)—, —NR′Z—(C═O)—, —NR′Z—O(C═O)—, —NR′Z—NH(C═O)—, —NR′Z—NR′(C═O)—, —NR′Z—S(C═O)—, —NR′Z—(C═NH)—, —NR′Z—O(C═NH)—, —NR′Z—NH(C═NH)—, —NR′Z—NR′(C═NH)—, —NR′Z—S(C═NH)—, —NR′Z—(C═NR′)—, —NR′Z—O(C═NR′)—, —NR′Z—NH(C═NR′)—, —NR′Z—NR′(C═NR′)—, —NR′Z—S(C═NR′)—, —SZ—(C═O)—, —SZ—O(C═O)—, —SZ—NH(C═O)—, —SZ—NR′(C═O)—, —SZ—S(C═O)—, —SZ—(C═NH)—, —SZ—O(C═NH)—, —SZ—NH(C═NH)—, —SZ—NR′(C═NH)—, —SZ—S(C═NH)—, —SZ—(C═NR′)—, —SZ—O(C═NR′)—, —SZ—NH(C═NR′)—, —SZ—NR′(C═NR′)—, —SZ—S(C═NR′)—, -J-O(C═O)—, —O-J-O(C═O)—, —S-J-O(C═O)—, —NH-J-O(C═O)—, —NR′-J-O(C═O)—, a polyether e.g. poly(alkylene glycol) having a molecular weight of from 76 to 2000 Da, a polyamine having a molecular weight of from 75 to 2000 Da, a polyester having a molecular weight of from 116 to 2000 Da, a polyamide having a molecular weight of from 114 to 2000 Da, and a moiety —W— wherein H—W—OH is an amino acid or a peptide containing from two to twenty naturally-occurring or synthetic amino acid subunits;

Z is selected from C₁₋₂₀ alkylene, C₁₋₂₀ alkenylene, C₁₋₂₀ alkynylene, C₆₋₁₀ arylene (e.g. phenylene or naphthylene), C₇₋₂₀ aralkylene, C₃₋₁₀ cycloalkylene, C₄₋₈ heterocycloalkylene, C₅₋₁₀ heteroarylene, and C₆₋₂₀ heteroaralkylene;

J is a phenyl group which carries a sugar substituent and, para or ortho to the sugar substituent, a methylene group or a moiety —(CH═CH)ₖ—CH₂—, wherein k is an integer from 1 to 10, further wherein the methylene group or moiety —(CH═CH)ₖ—CH₂— is directly bonded to the —O(C═O)— group proximal to the biologically active moiety B¹, B² or B³, and a carbon of the phenyl ring is directly bonded to the remainder of the linker group distal to the biologically active moiety B¹, B² or B³;

each K is the same or different and represents C₁₋₁₀ alkylene;

i is an integer from 1 to 100, preferably from 1 to 50, and more preferably from 2 to 20;

v is an integer from 0 to 4, preferably 1 or 2; and

R′ is C₁₋₂₀ hydrocarbyl.

More preferably, the linker group is ═N—O—Z¹-L′-Z²—, wherein Z¹ and L′ are as defined above and Z² is selected from —Z—(C═O)—, —Z—O(C═O)—, —Z—NH(C═O)—, —Z—NR′(C═O)—, —Z—S(C═O)—, —OZ—(C═O)—, —OZ—O(C═O)—, —OZ—NH(C═O)—, —OZ—NR′(C═O)—, —OZ—S(C═O)—, —NHZ—(C═O)—, —NHZ—O(C═O)—, —NHZ—NH(C═O)—, —NHZ—NR′(C═O)—, —NHZ—S(C═O)—, —NR′Z—(C═O)—, —NR′Z—O(C═O)—, —NR′Z—NH(C═O)—, —NR′Z—NR′(C═O)—, —NR′Z—S(C═O)—. SZ—(C═O)—, —SZ—O(C═O)—, —SZ—NH(C═O)—, —SZ—NR′(C═O)—, —SZ—S(C═O)—, -J-O(C═O)—, —O-J-O(C═O)—, —S-J-O(C═O)—, —NH-J-O(C═O)—, —NR′-J-O(C═O)—, a polyester having a molecular weight of from 116 to 2000 Da, a polyamide having a molecular weight of from 114 to 2000 Da, and a moiety —W—, or, when L′ is a moiety —W—, Z² may additionally be a bond. Preferably, the linker group is ═N—O—Z¹-L′-Z²— and the end of the linker distal to the ═N—O— moiety terminates in a carbonyl group.

A particularly preferred linker group is selected from ═N—NH—CH₂—(C═O)-Val-Cit-PAB-(C═O)—, ═NO—CH₂—(C═O)-Val-Cit-PAB-(C═O)—, ═N—CH₂—(C═O)-Val-Cit-PAB-(C═O)—, —NH—NH—CH₂—(C═O)-Val-Cit-PAB-(C═O)—, —NHO—CH₂—(C═O)-Val-Cit-PAB-(C═O)— and —NH—CH₂—(C═O)-Val-Cit-PAB-(C═O)—, wherein -Val-Cit-PAB- has the following structure:

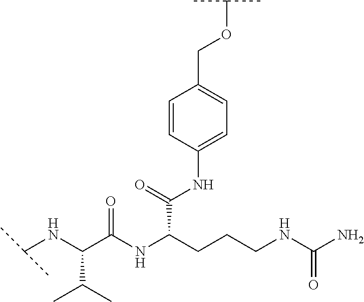

This is a well-known linker group in the field of polymer-drug conjugates.

Most preferably, the linker group is ═NO—CH₂—(C═O)-Val-Cit-PAB-(C═O)—.

Preferably, the moiety J is a phenyl group which carries a methylene group para or ortho to the sugar substituent. More preferably, the methylene group is para to the sugar substituent. Even more preferably, the sugar substituent in the moiety J is bound to the phenyl group via an oxygen atom that is also directly bonded to the anomeric carbon atom of the sugar. Yet more preferably, the sugar substituent is a six-carbon sugar. Still more preferably, the sugar substituent is selected from a sugar substituent which can be converted to a hydroxyl substituent by the action of an enzyme, such as glucuronic acid (which can be cleaved by the action of β-glucuronidase). Most preferably, the moiety J has the following structure:

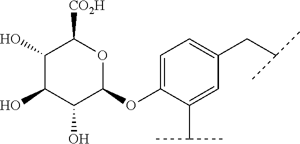

A particularly preferred linker group comprising a moiety J is selected from the following structures:

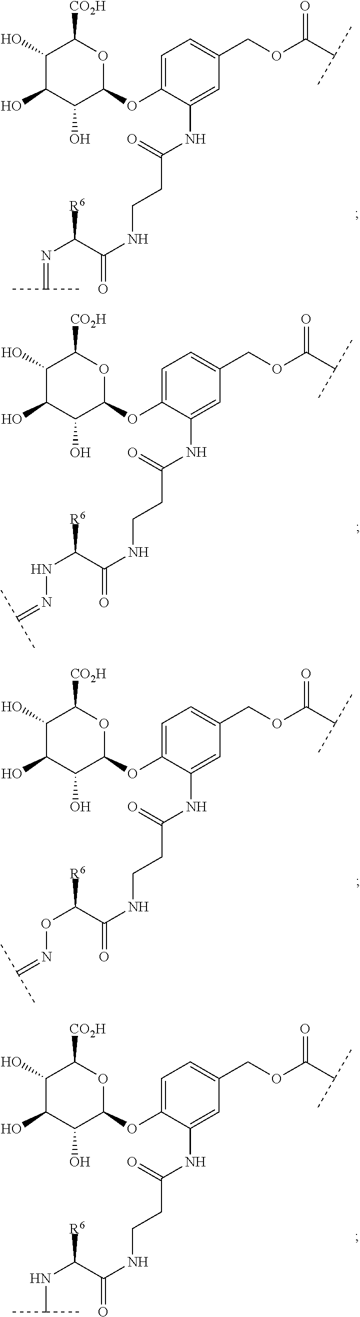

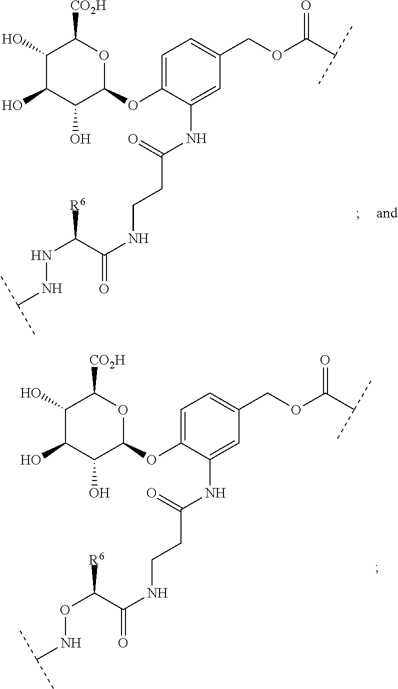

wherein R⁶ is selected from any amino acid R group or derivative thereof, e.g. H, CH₃, CH(CH₃)₂, CH₂CH(CH₃)₂, CH(CH₃)CH₂CH₃, CH₂Ph, CH₂NH₂, CH₂OH, CH₂SH, CH(OH)CH₃, CH₂CH₂SCH₃, CH₂CONH₂, CH₂CH₂CONH₂, CH₂COOH, CH₂CH₂COOH, (CH₂)₃NH(CN)NH₂, (CH₂)₄NH₂, (CH₂)₃NH₂,

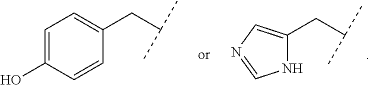

Preferably, R⁶ is selected from H, CH₃ and CH₂NH₂, and is more preferably CH₂NH₂.

Polymer-drug conjugates having a linker group selected from —NH—NH—Z¹-L′-Z²—, —NH—O—Z¹-L′-Z²— and —NH—Z¹-L′-Z²— may be obtained by the reduction of polymer-drug conjugates having a linker of formula ═N—NH—Z¹-L′-Z²—, ═N—O—Z¹-L′-Z²— or ═N—Z¹-L′-Z²—, respectively.

For the avoidance of doubt, in the above definitions of a linker group, the left-hand side of the linker group as drawn (the top of the linker group as drawn for the cyclic acetal and cyclic thioacetal) attaches to the polymer backbone of the antibody-drug conjugate, and the right-hand side of the linker group as drawn (the bottom of the linker group as drawn for the cyclic acetal and cyclic thioacetal) attaches to the biologically active moiety B¹, B² or B³. In the above depiction of the linker -Val-Cit-PAB-, the left-hand side shows the external bond to valine (Val) and the top shows the external bond to para-amino benzyl alcohol (PAB). In the above depiction of preferred linker groups comprising a moiety J, the bottom left shows the attachment to the polymer backbone, and the top right shows the attachment to the biologically active moiety B¹, B² or B³.

Typically, L′ has a molecular weight of from 14 to 2000 Da, preferably from 28 to 1000 Da, more preferably from 50 to 500 Da, and yet more preferably from 100 to 300 Da. Typically, Z¹ has a molecular weight of from 14 to 2000 Da, preferably from 28 to 1000 Da, more preferably from 50 to 500 Da, and yet more preferably from 100 to 300 Da. Typically, Z² has a molecular weight of from 14 to 2000 Da, preferably from 28 to 1000 Da, more preferably from 50 to 500 Da, and yet more preferably from 100 to 300 Da.

When L¹, L² or L³ is a linker group, L¹, L² or L³ is typically derived from a compound of formula HL¹-LG, HL²-LG or HL³-LG. LG is a leaving group under addition-elimination reaction conditions. Addition-elimination conditions are well-known to a person skilled in the art. Typically, addition-elimination conditions are any reaction conditions under which a nucleophilic (i.e. electron-rich) moiety can add to an unsaturated carbon atom to form a covalent σ-bond to that carbon atom, resulting in the disruption of a π-bond to the carbon atom, and the subsequent re-formation of said π-bond and the concomitant breaking of a σ-bond between said carbon atom and one of its other substituents, which is typically a net electron-withdrawing moiety, to eliminate that substituent. Preferably, LG is selected from halo (preferably Cl), OH, OR′, SH, SR′, NH₂, NHR′, NR′₂, O-2-Cl-Trt, ODmb, O-2-PhⁱPr, O-EDOTn-Ph, OFm, ODmab and OCam, wherein R′, 2-Cl-Trt, Dmb, EDOTn-Ph, Fm, Dmab and OCam are as defined above.

Typically, the bond(s) between either the polymer repeat unit or the linker unit and B¹, B² or B³ is/are acid-labile. Preferably in this case, the bond(s) is/are hydrolysed in the acidic and/or hydrolytic environment of cell compartments such as lysosome, endosome, phagosome, phagolysosome and autophagosome found in various cells such as macrophages. Preferably in this case, the bond(s) between either the polymer repeat unit or the linker unit and B¹, B² or B³ is/are hydrolysed in a pH of <6 and still more preferably in a pH of <5. An example of a bond hydrolysed in an acidic environment is a hydrazone bond.

Alternatively, the bond(s) between either the polymer repeat unit or the linker unit and B¹, B² or B³ is/are labile in neutral conditions. Preferably in this case, the bond(s) between either the polymer repeat unit or the linker unit and B¹, B² or B³ is/are hydrolysed at a neutral pH, preferably a pH of from 6.5 to 7.5.

Alternatively, the bond(s) between either the polymer repeat unit or the linker unit and B¹, B² or B³ is/are base-labile. Preferably the bond(s) between either the polymer repeat unit or the linker unit and B¹, B² or B³ is/are hydrolysed at a pH of >8 and still more preferably in a pH of >9.

The optimum pH at which the bond(s) is/are hydrolysed will depend on the precise chemical nature of the relevant bond(s).

Alternatively, the bond(s) between either the polymer repeat unit or the linker unit and B¹, B² or B³ is/are hydrolysed in the presence of an enzyme. Preferably in this case, the bond(s) between either the polymer repeat unit or the linker unit and B¹, B² or B³ is/are hydrolysed by cathepsin B. An example of a bond hydrolysed enzymatically by cathepsin B is a peptide bond.

Alternatively, the bond(s) between either the polymer repeat unit or the linker unit and B¹, B² or B³ is/are resistant to hydrolysis. For example, the bond(s) between either the polymer repeat unit or the linker unit and B¹, B² or B³ may be cleaved through disulfide exchange with an intracellular thiol (e.g. glutathione). An example of a bond that can be cleaved in this manner is a disulfide bond. Alternatively, the bond(s) between either the polymer repeat unit or the linker unit and B¹, B² or B³ may be cleaved through intracellular proteolytic degradation. An example of a bond that can be cleaved in this manner is a thioether bond.

The cleavage of the bond(s) between either the polymer repeat unit or the linker unit and B¹, B² or B³ releases the said biologically active molecule (e.g. a drug). Preferably, there is a linker group between the polymer repeat unit and the moiety B¹, B² or B³. More preferably the bond between the repeat unit and the linker moiety L¹, L² or L³ is a double bond. Alternatively, L¹, L² or L³ is/are a bond, i.e. there is a direct bond between the polymer repeat unit and the moiety B¹, B² or B³. In this case, preferably the bond between the repeat unit and the moiety B¹, B² or B³ is a double bond.

Preferably the biologically active molecule from which the polymer repeat unit is derived comprises a functional group that is able to form a covalent bond with a keto group present in the compound of Formula (IIa), (IIa′), (IIc) or (IIc′), or with a carbonyl group present in the linker L¹, L² or L³. More preferably the biologically active molecule (e.g. a drug) comprises at least one hydrazine group, at least one hydrazide group, at least one amine group, at least one aminooxy group, at least one, preferably two, hydroxyl groups or at least one, preferably two, thiol groups. Even more preferably, the biologically active molecule from which the polymer repeat unit is derived is able to form an amide bond, an ester bond, a carbamate bond or a carbonate bond with a carbonyl group present in the linker moiety L¹, L² or L³, preferably an amide bond. Alternatively, the biologically active molecule from which the polymer repeat unit is derived comprises a functional group that is able to form a covalent bond with an amino, hydroxyl or thiol group present in the linker L¹, L² or L³. In this case, preferably the biologically active molecule (e.g. a drug) comprises at least one carboxylic acid group or at least one thiol group. Even more preferably, the biologically active molecule from which the polymer repeat unit is derived is able to form an ester bond with a hydroxyl group present in the linker moiety L¹, L² or L³, an amide bond with an amino group present in the linker moiety L¹, L² or L³, a thioester bond with a thiol group present in the linker moiety L¹, L² or L³, or a disulfide bond with a thiol group present in the linker moiety L¹, L² or L³.

More preferably, each D¹ and D³ is O. Thus, preferably at least one D² is L²-B². In one aspect, each D² is L²-B².

In one aspect, each D¹ and D³ is O, at least one D² is L²-B², and the bond between the polymer backbone and D² is a hydrazone. Preferably therefore the polymer comprises a repeat unit of Formula (IA), e.g. a repeat unit of Formula (Ia).

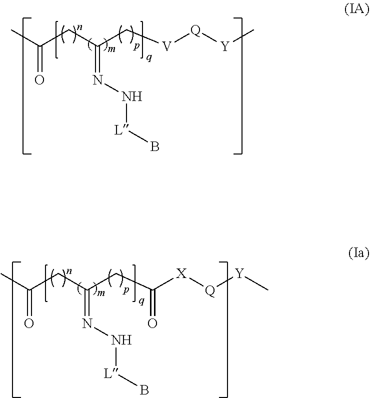

wherein n, m, p, q, V, X, Y and Q are as defined above in relation to Formula (I′), (I) or (I″), L″ is a bond or —Z¹-L′-Z²— as defined above, and B is either the biologically active moiety B², or, if L″ is a bond, B may alternatively be defined such that B—NH—N is the biologically active moiety B². Preferred identities for each of n, m, p, q, X, Y and Q are as set out above in relation to each of Formulae (I′), (I), (I″), (IIa), (IIa′), (IIb), (IIb′), (IIc) and (IIc′).

When L″ is a bond and the biologically active moiety is released by hydrolysis of the C═N bond in vivo, the biologically active molecule is B—NHNH₂. Advantageously the hydrazone bond hydrolyses at a pH of <6. In preferred polymers comprising a repeat unit of Formula (IA) or (Ia), the biologically active molecule is a drug is selected from isoniazid, carbidopa, endralazine, dihydralazine, hydralazine, hydracarbazine, pheniprazine, pildralazine, octamoxin, a synthetic peptide, a synthetic oligonucleotide, a carbohydrate, a peptide mimetic, an antibody, hydrazine and mixtures thereof.

In another aspect, the hydrazone bond in Formula (IA) or (Ia) may further be reduced. Reduction of a hydrazone bond may be effected by any known technique in the art. Preferably, in this aspect, the polymer comprises a repeat unit of Formula (IA′), e.g. a repeat unit of Formula (Ia′):

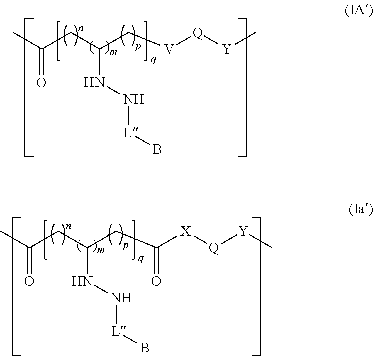

wherein all of the variables are as defined above in relation to Formula (IA) or (Ia).

In another aspect, each D¹ and D³ is O, at least one D² is L²-B², and the bond between the polymer backbone and D² is an imine. Preferably therefore the polymer comprises a repeat unit of Formula (IB), e.g. a repeat unit of Formula (Ib):

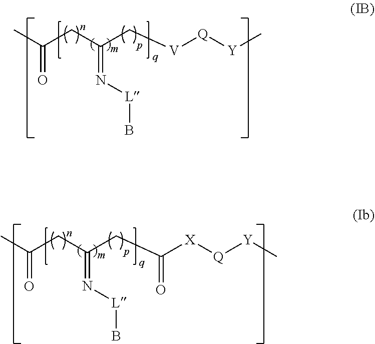

wherein n, m, p, q, X, Y and Q are as defined above in relation to Formula (I′), (I) or (I″), L″ is a bond or —Z¹-L′-Z²— as defined above, and B is either the biologically active moiety B², or, if L″ is a bond, B may alternatively be defined such that B—N is the biologically active moiety D². Preferred identities for each of n, m, p, q, X, Y and Q are as set out above in relation to each of Formulae (I′), (I), (I″), (IIa), (IIa′), (IIb), (IIb′), (IIc) and (JIc′).

When L″ is a bond and the biologically active moiety is released by hydrolysis of the C═N bond in vivo, the biologically active molecule is B—NH₂. In preferred polymers comprising a repeat unit of Formula (IB) or (Ib), the biologically active molecule is selected from Alteplase, Adalimumab, Bivalirudin, Chloroprocaine, Daptomycin, Doxazosin, Efavirenz, Hydroflumethiazide, Indapamide, Insulin Detemir, Lisinopril,peptide mimetics, Prazosin, Saxagliptin, small interfering RNA, Sulfamethylthiazole, Sulfametrole, Sulfisomidine, Tripamide, 2-p-Sulfanilylanilinoethanol, 3-Amino-4-hydroxybutyric Acid, 3-Aminopyridine-2-carboxaldehyde thiosemicarbazone (3-AP)/3-Aminopyridine-4-methyl-2-carboxaldehyde thiosemicarbazone (3-AMP/Triapine/OCX-191/OCX-0191), 4, 4′-Sulfinyldianiline, 4′-(Methylsulfamoyl)sulfanilanilide, 4′-Sulfanilylsulfanilamide, 4-Amino-3-hydroxybutyric Acid, 4-Sulfanilamidosalicylic acid, 5-Hydroxytryptophan, 6-Diazo-5-oxo-L-norleucine (DON), 9-Aminoacrindine, 9-Aminocamptothecin, Abacavir, Abatacept, Acediasulfone, Acetosulfone sodium, Acyclovir, Adefovir, Alfuzosin, Amantadine, Amfenac, Amidinomycin, Amikacin, Aminolevulinic Acid, Amlodipine, Amoxicillin, Amphetamine, Amphomycin, Amphotericin B, Ampicillin, Amprenavir, Ancitabine, antibodies, antigens, Arbekacin, Aspoxicillin, Azacitidine, Azaserine, Bacampicillin, Bacitracin, BenexateHCl, Benserazide, Benzocaine, Benzylsulfamide, Bevacizumab, Bleomycins, Brodioprim, Bropirimine, Bunazosin, Butirosin, Capreomycin, carbohydrates, Carboplatin, Carubicin, Carumonam, Caspofungin, Cefaclor, Cefadroxil, Cefatrizine, Cefcapene, Cefclidin, Cefdinir, Cefditoren, Cefepime, Cefetamet, Cefinenoxime, Cefixime, Cefminox, Cefodizime, Ceforanide, Cefoselis, Cefotaxime, Cefotiam, Cefozopran, Cefpirome, Cefpodoxime, Cefprozil, Cefroxadine, Ceftazidime, Cefteram, Ceftibuten, Ceftizoxime, Ceftriaxone, Cefuzonam, Celecoxib, Cephalexin, Cephaloglycin, Cephalosporin C, Cephradine, Certolizumab, Cetoxime, Cetraxate, Cetuximab, Chlorproguanil, Cidofovir, Cilastatin, Cladribine, Clinafloxacin, Clopamide, Colesevelam, Colistin, Cyclacillin, Cycloguanil, Cyclopenthiazide, Cycloserine, Cytarabine, Dapsone, Darbepoetin Alfa, Darunavir, Daunorubicin, Decitabine, Denosumab, Dextroamphetamine, Dezocine, Dibekacin, Dideoxyadenosine, Disoproxil, DNA, Dornase Alfa, Doxorubicin, Doxycycline, Ebrotidine, Edatrexate, Eflornithine, Emtricitabine, Entecavir, Enviomycin, Epicillin, Epinastine, Epirubicin, Epoetin Alfa, Etanercept, Ethambutol, Exenatide, Famciclo Imiquimodvir, Famotidine, Filgrastim, Fingolimod, Flucytosine, Fluvoxamine, foldamers, Folic acid, Forimicins, Gabapentin, gama-Aminobutyric acid, Gemcitabine, Gemifloxacin, Gentamicin, Glatiramer Acetate, Golimumab, Histamine, Human Papilloma Quadrivalent, Hydrochlorothiazide, Idarubicin, Immune Globulin, Infliximab, Insulin Aspart, Insulin Glargine, Insulin Lispro, Interferon beta-1a, Interferon beta-ib, Ipilimubab, Irsogladine, Isepamicin, Kanamycin(s), Lamivudine, Lamotrigine, Lanreotide, L-DOPA, Lenalidomide, Lenampicillin, Levodopa, Levothyroxine, Liraglutide, Lisdexamfetamine, Loracarbef, Lymecycline, Mafenide, Mantadine, Meclocycline, Melphalan, Memantine, Mesalamine, Mesalazine, Metformin, Methacycline, Methotrexate, Methyl Aminolevulinate, Methyldopa, Miboplatin, Micronomicin, microRNA, Mikamycin, Milnacipran, Minocycline, Mitoguazone, Morphazinamide, mRNA, N4-beta-D-Glucosylsulfanilamide, Natalizumab, Natamycin, Negamycin, Neomycin, Netilmicin, Nimustine, Nolatrexed, Nomifensine, Non-Lipinski molecules, Noprysulfamide, N-Sulfanilyl-3, 4-xylamide, Nystatin, Ocreotide Acetate, Omalizumab, Oseltamivir, Oxaliplatin, Palivizumab, p-Aminosalicylic acid, p-Aminosalicylic acid hydrazide, Paromomycin, Parsalmide, Pazufloxacin, Pegfilgrastim, Peginterferon alfa-2a, Pemetrexed, Penciclovir, Peplomycin, Peptide, Protein, Pexiganan, Phenyl aminosalicylate, Picloxydine, Pirarubicin, Piritrexim, Pivampicillin, Pivcefalexin, pivoxil, PNA, Polymyxin, Pralatrexate, Pregabalin, Pregabelin, Primaquine, Procaine, Proparacaine, Propoxycaine, Proxetil, p-Sulfanilylbenzylamine, Puromycin, pyrimethamine, Quinocide, Ramoplanin, Ranibizumab, Regadenoson, Remacemide, Resiquimod, Ribostamycin, Rimantadine, Ristocetin, Rituximab, Rotraxate, S-Adenosylmethionine, Salacetamide, Sampatrilat, Sevelamer, Sisomicin, Sitafloxacin, Sitagliptin, small hairpin RNA, S-Methylmethionine, Somatropin, Sparfloxacin, Streptonigrin, Succisulfone, Suclofenide, Sulfabenzamide, Sulfacetamide, Sulfachlorpyridazine, Sulfachrysoidine, Sulfacytine, Sulfadiazine, Sulfadicramide, Sulfadimethoxine, Sulfadoxine, Sulfaethidole, Sulfaguanidine, Sulfaguanole, Sulfalene, Sulfamerazine, Sulfameter, Sulfamethazine, Sulfamethizole, Sulfamethoxazole, ulfamethoxypyridazine, Sulfamidochrysoidine, Sulfamoxole, Sulfanilamide, Sulfanilic acid, Sulfanilylurea, Sulfaperine, Sulfaphenazole, Sulfaproxyline, Sulfapyrazine, Sulfasomizole, Sulfasymazine, Sulfathiazole, Sulfathiourea, Sulfatolamide, Sulfisoxazole, Sulfonamide, Sulframethomidine, Sultamicillin, Sulthiame, synthetic oligonucleotides, synthetic peptide, Tafenoquine, Talampanel, Talampicillin, Teicoplanin, Tenofovir, Terazosin, Teriparatide, Tetroxoprim, Thiamiprine, Thioguanine, Tigemonam, Tinoridine, Tirapazamine, Tobramycin, Topiramate, Tosufloxacin, Tranylcypromine, Trastuzumab, Trimazosin, Trimethoprim, Trimetrexate, Tritoqualine, Trovafloxacin, Troxacitabine, Tuberactinomycin, Tubercidin, Tyrocidine, Ustekinumab, Valacyclovir, Valdecoxib, Valganciclovir, Vancomycin, Vidarabine, Vigabatrin, Vindesine, Viomycin, Zalcitabine, Zonisamide, and mixtures thereof.

In another aspect, the imine bond in Formula (IB) or (Ib) may further be reduced. Reduction of an imine bond may be effected by any known technique in the art. Preferably, in this aspect, the polymer comprises a repeat unit of Formula (IB′), e.g. a repeat unit of Formula (Ib′):

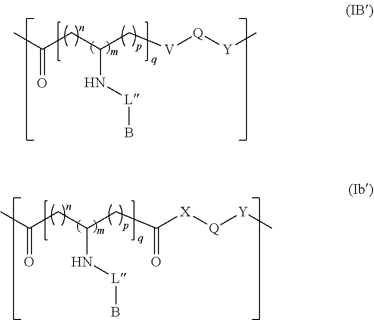

wherein all of the variables are as defined above in relation to Formula (IB) or (Ib).

In another aspect, each D¹ and D³ is O, at least one D² is L²-B², and the bonds between the polymer backbone and D² form a ketal. Other particularly preferred polymers comprise two biologically active moieties, and the bonds between the polymer backbone and D² form a ketal. Preferably therefore the polymer comprises a repeat unit of Formula (ICi) or (ICii), e.g. a repeat unit of Formula (Ici) or (Icii):

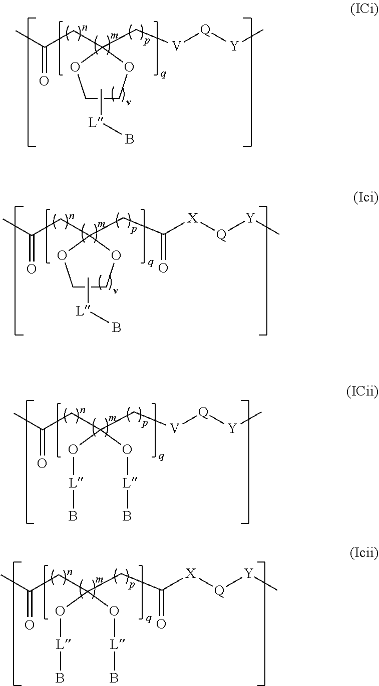

wherein n, m, p, q, v, V, X, Y and Q are as defined above in relation to Formula (I′), (I) or (I″), L″ is a bond or —Z¹-L′-Z²— as defined above, and B is either the biologically active moiety B², or, if L″ is a bond, B may alternatively be defined such that B—O or O—B—O is the biologically active moiety B². Preferred identities for each of n, m, p, q, v, V, X, Y and Q are as set out above in relation to each of Formulae (I), (I′), (I″), (IIa), (IIa′), (IIb), (IIb′), (IIc) and (IIc′). When L″ is a bond and the biologically active moiety/moieties is/are released by hydrolysis of the C—O bond(s) in vivo, the biologically active molecule(s) is/are B—OH or HO—B—OH.

In preferred polymers comprising a repeat unit of Formula (ICi), (Ici), (ICii) or (Icii), the biologically active molecule is selected from 2,4,6-Tribromo-m-cresol, 21-Acetoxypregnenolone, 2-p-Sulfanilylanilinoethanol, 3-Amino-4-hydroxybutyric Acid, 4-Amino-3-hydroxybutyric Acid, 4-Hexylresorcinol, 4-Sulfanilamidosalicylic acid, 5-(methylamino)-2-deoxyuridine (MADU), 5-Bromosalicylhydroxamic acid, 5-Hydroxytryptophan, 9-Aminocamptothecin, Abacavir, Abatacept, Abiraterone, Acebutolol, Acetaminophen, Acetaminosalol, Aclacinomycins, Acyclovir, Adalimumab, Ajmaline, Alclometasone, alfa-Bisabolol, all erythromycin ester derivatives, Alprenolol, Alteplase, Aluminum bis(acetylsalicylate), Amikacin, Aminochlorthenoxazin, Aminopropylon, amodiaquine, Amosulalol, Amoxicillin, Amprenavir, Ancitabine, Anidulafungin, Anileridine, Anthramycin, antibodies, antigens, Apalcillin, Apicycline, Arbekacin, Arotinolol, Artemisinin alcohol, Arzoxifene, Aspoxicillin, Atazanavir, Atenolol, Atrolactamide, Azacitidine, Azidamfenicol, Azithromycin, Bambermycins, Batimastat, Bebeerines, Beclomethasone Dipropionate, Befloxatone, Benserazide, Benzoylpas, Benzylmorphine, Betamethasone, Betaxolol, Bevacizumab, Biapenem, Bimatoprost, Bisoprolol, Bleomycins, Bosentan, Bromosalicylchloranilide, Broxuridine, Bucetin, Bucindolol, Budesonide, Bufeniode, Bufexamac, Bunitrolol, Bupranolol, Buprenorphine, Bupropion, Buramate, Buserelin, Butirosin, Butofilolol, Butorphanol, Cadralazine, Calusterone, Capecitabine, Capreomycin, Capsaicine, Carazolol, Carbidopa, carbohydrates, Carbomycin, Carteolol, Carubicin, Carvedilol, Caspofungin, CC-1065, Cefadroxil, Cefamandole, Cefatrizine, Cefbuperazone, Cefonicid, Cefoperazone, Cefoselis, Cefpiramide, Cefprozil, Celiprolol, Cephapirin sodium, Certolizumab, Cetuximab, Chloramphenicol, Chlorobutanol, Chloroxylenol, Chlorozotocin, Chlorphenesin, Chlorquinadol, Chlortetracycline Dalfopristin, Chromomycins, Cicletanine, Ciclopirox, Ciclosporine, Cidofovir, Cinchonidine, Cinchonine, Ciramadol, Cladribine, Clarithromycin, clavulanic acid, Clindamycin, Clobetasone, Clofoctol, Clomocycline, Cloxyquin, Codeine, Colesevelam, Colistin, Cyclosporin, Cytarabine, Darbepoetin Alfa, Darunavir, Dasatinib, Daunorubicin, Decitabine, Deflazacort, Delmostatin, Demeclocycline, Denosumab, Deoxydihydrostreptomycin, Desomorphine, Desonide, Desoximetasone, Desvenlafaxine, Dexamethasone, Dezocine, Diathymosulfone, Dibekacin, Didanosine, Dideoxyadenosine, Diethylstilbestrol, Diflorasone, Diflucortolone, Diflunisal, Gentisic acid, Difluprednate, Dihydroartemisinin, Dihydrocodeine, Dihydromorphine, Dihydrostreptomycin, Dihydroxyaluminum acetylsalicylate, Dilevalol, Dimepheptanol, Dirithromycin, Ditazol, DNA, Docetaxel, Dornase Alfa, Doxifluridine, Doxorubicin, Doxycycline, Droloxifene, Dromostanolone, Ecteinascidins, Edoxudine, Emtricitabine, Enocitabine, Enoxaparin, Enoxolone, Enprostil, Entacapone, Entecavir, Enviomycin, Epanolol, Epinephrine, Epirubicin, Epitiostanol, Epoetin Alfa, Eptazocine, Ertapenem, Erythromycin, Estramustine, Etanercept, Etanidazole, Ethinyl Estradiol, Ethoxazene, Ethylmorphine, Etofenamate, Etonogestrel, Etoposide, Eugenol, Everolimus, Exenatide, Ezetimibe, Fendosal, Fenoldopam Fenpentadiol, Fenretinide, Fepradinol, Fexofenadine, Filgrastim, Filipin, Flavopiridol, Flipirtine, Floctafenine, Flomoxef, Floxuridine, Fluazacort, Fluconazole, Fludrocortisone, Flumethasone, Fluocinolone, Fluocinonide, Fluocortin Butyl, Fluocortolone, Fluprednidene Acetate, Fluticasone Propionate, foldamers, Forimicins, Formestane, Formoterol, Foscarnet sodium, Fosfestrol, Fropenem, Fulvestrant, Fungichromin, Furonazide, Fusidic acid, Galantamine, Ganciclovir, Gemcitabine, Gentamicin, Glafenine, Glucametacin, Glucosulfone sodium, Glyconiazide, Golimumab. Balsalazide, Goserelin, Gramicidin(s), Guamecycline, Halcinonide, Halobetasol Propionate, Halofantrine, Halometasone, Halopredone Acetate, Human Papilloma Quadrivalent, Hydrocortisone, Hydromorphone, Hydroxypethidine, Hypericin, Ibuproxam, Idarubicin, Idoxuridine, Imipenem, Immune Globulin, Indenolol, Indinavir, Infliximab, Insulin Aspart, Insulin Detemir, Insulin Glargine, Insulin Lispro, Interferon beta-1a, Interferon beta-lb, Ipilimubab, Ipratropium, Irinotecan, Isepamicin, Isoxicam, Kanamycin(s), Kethoxal, Ketobemidone, Labetalol, Lamivudine, Latanoprost, L-DOPA, Leuprolide, Levcromakalim, Levodopa, Levonorgestrel, Levorphanol, Levothyroxine, Lincomycin, Liraglutide, Lopinavir, Lornoxicam, Losartan, Loteprednol Etabonate, Lumefantrine, Lymecycline, Mannomustine, Marimastat, Mazipredone, Meclocycline, Mefloquine, Melengestrol, Meloxicam, Memetasone, Menogaril, Mepindolol, Meptazinol, Merbromin, Meropenem, Mesalamine, Mesalazine, Metazocine, Methacycline, Methyldopa, Methylprednisolone, Metipranolol, Metopon, Metoprolol, Metronidazole, Micronomicin, microRNA, Mikamycin, Miltefosine, Minocycline, Misoprostol, Mitobronitol, Mitolactol, Mitoxantrone, Mometasone Furoate, Montelukast, Mopidamol, Moprolol, Morphine, Moxalactam, mRNA, N4-beta-D-Glucosylsulfanilamide, Nadifloxacin, Nadolol, Naftopidil, Nalbuphine, Natalizumab, Nebivolol, Negamycin, Nelfinavir, Neomycin, Netilmicin, N-Hydroxyethylpromethazine Chloride, Nifurpirinol, Nifurtoinol, Nitracrine, Nitroxoline, Nogalamycin, non-Lipinski molecules, Nordihydroguaiaretic Acid, Norlevorphanol, Normorphine, Novobiocin, Oleandomycin, Olivomycins, Olmesartan, Olsalazine, Omalizumab, Opipramol, Ornoprostil, Oryzanol A. Ganaxolone, Oxaceprol, Oxametacine, Oxycodone Pentazocine, Oxycodone, Oxymorphone, Oxyphenbutazone, Oxytetracycline, Paclitaxel and other known paclitaxel analogs, Paclitaxel, Paliperidone Palmitate, Paliperidone, Palivizumab, p-Aminosalicylic acid hydrazide, p-Aminosalicylic acid, Panipenem, Paromomycin, Pecilocin, Pegfilgrastim, Peginterferon alfa-2a, Penbutolol, Penciclovir, Pentostatin, Peplomycin, peptide mimetics, peptide, Perisoxal, Phenactropinium chloride, Phenazocine, Phenazopyridine, Phenocoll, Phenoperidine, Phentolamine, Phenyl aminosalicylate, Phenylramidol, Phenylsalicylate, Pildralazine, Pimecrolimus, Pindolol, Pipacycline, Pirarubicin, Piroxicam, p-Lactophenetide, Plaunotol, Plicamycin, PNA, Podophyllotoxin, Polymyxin, Posaconazole, Prednisolone, Prednisone, Primycin, Pristinamycin, Propranolol, protein, Protoveratrines, Puromycin, Pyrisuccideanol, Quetiapine, Ezetimibe, Quinine, Quinupristin, Raloxifene, Raltegravir, Ramoplanin, Ranibizumab, Ranimustine, Ranolazine, Ravuconazole, Rescimetol, Resiquimod, Retinoic acid (including all trans-retinioc acid), Ribavirin, Ribostamycin, Rifabutin, Rifalazil, Rifamide, Rifampicin, Rifamycin SV, Rifapentine, Rifaximin, Rimexolone, Rioprostil, Risedronic Acid, Ristocetin, Ritipenem, Ritonavir, Rituximab, Rolitetracycline, Roquinimex, Rosaprostol, Roxarsone, Roxindole, Roxithromycin, Rubijervine, Rubitecan, S-Adenosylmethionine, Salazosulfadimidine, Salicin, Tramadol, Salicylamide, Salicylanilide, Salinazid, Salmeterol, Salsalate, Sampatrilat, Sancycline, Saquinavir, Saxagliptin, Seocalcitol, Sevelamer, Siccanin, Simvastatin, Sirolimus, Sisomicin, small hairpin RNA, small interfering RNA, Somatropin, Sorivudine, Spectinomycin, Stavudine, Streptolydigin, Streptomycin, Streptonicozid, Streptozocin, Sulfasalazine, Sulfinalol, synthetic oligonucleotides, synthetic peptide, Tacrolimus, Tacrolimus. Talinolol, Teicoplanin, Telithromycin. Temoporfin, Teniposide, Tenoxicam, Tenuazonic Acid, Terfenadine, Teriparatide, Terofenamate, Tertatolol, Testosterone, Thiamphenicol, Thiostrepton, Tiazofurin, Timolol, Tiotropium, Tipranavir, Tobramycin, Tolcapone, Toloxatone, Tolterodine, Topotecan, Trans-Resveratrol [(E)-3,4′,5-trihydroxystilbene), Trastuzumab, Travoprost, Triamcinolone, Trifluridine, Trimazosin, Trimoprostil, Trospectomycin, Troxacitabine, Tuberactinomycin, Tyrocidine, Ustekinumab, Valdecoxib, Valganciclovir, Valrubicin, Vancomycin, Venlafaxine, Vidarabine, Viminol, Vinblastine, Vincristine, Vindesine, Viomycin, Virginiamycin, Voriconazole, Xanthocillin, Xibomol, Ximoprofen, Yingzhaosu A, Zalcitabine, Zanamivir, Zidovudine, Zoledronic Acid, Zolendronic Acid, Zorubicin, Zosuquidar, and mixtures thereof.

In another aspect, each D¹ and D³ is O, at least one D² is L²-B², and the bonds between the polymer backbone and D² form a thioketal. Other particularly preferred polymers comprise two biologically active moieties, and the bonds between the polymer backbone and D² form a thioketal. Preferably therefore the polymer comprises a repeat unit of Formula (IDi) or (IDii), e.g. a repeat unit of Formula (Idi) or (Idii):

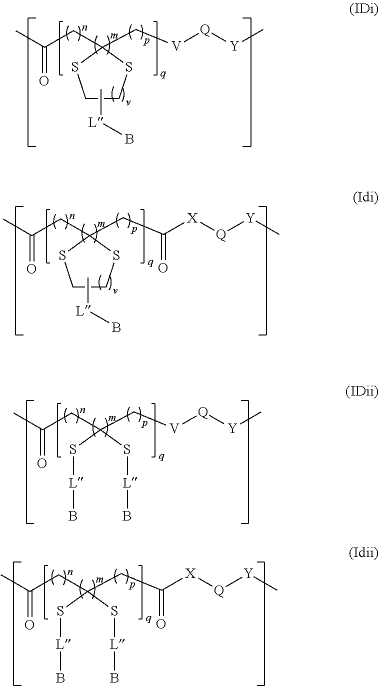

wherein n, m, p, q, v, V, X, Y and Q are as defined above in relation to Formula (I′), (I) or (I″), U″ is a bond or —Z¹-L′-Z²— as defined above, and B either the biologically active moiety B², or, if L″ is a bond, B may alternatively be defined such that B—S or S—B—S is the biologically active moiety B². Preferred identities for each of n, m, p, q, v, V, X, Y and Q are as set out above in relation to each of Formulae (I), (I′), (I″), (IIa), (IIa′), (IIb), (IIb′), (IIc) and (IIc′). When L″ is a bond and the biologically active moiety/moieties is/are released by hydrolysis of the C—O bond(s) in vivo, the biologically active molecule(s) is/are B—SH or HS—B—SH.

In preferred polymers comprising a repeat unit of Formula (IDi) or (IDii), e.g. a repeat unit of Formula (Idi) or (Idii), the biologically active molecule is selected from a peptide, protein, carbohydrate, peptide mimetic, antibody, antigen, synthetic oligonucleotide, Adalimumab, Etanercept, Pegfilgrastim, Rituximab, Bevacizumab, Insulin Glargine, Epoetin Alfa, Trastuzumab, Interferon beta-1a, Ranibizumab, Insulin Detemir, Insulin Aspart, Insulin Lispro, Filgrastim, Darbepoetin Alfa, Interferon beta-lb, Abatacept, Liraglutide, Palivizumab, Cetuximab, Ustekinumab, Denosumab, Human Papilloma Quadrivalent, Peginterferon alfa-2a, Ipilimubab, Immune Globulin, Dornase Alfa, Certolizumab, Natalizumab, Somatropin, Alteplase, Golimumab, and mixtures thereof.

Alternatively, each D¹ and D³ is O, at least one D² is L²-B², and the bond between the polymer backbone and D² is an oxime. Preferably therefore the polymer comprises a repeat unit of Formula (IE), e.g. a repeat unit of Formula (Ie)

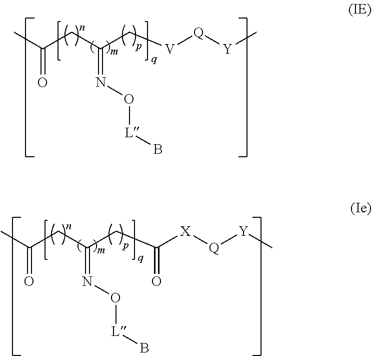

wherein n, m, p, q, V, X, Y and Q are as defined above in relation to Formula (I, (I′) or (I″), L″ is a bond or —Z¹-L′-Z²— as defined above, and B is either the biologically active moiety B², or, if L″ is a bond, B may alternatively be defined such that B—O—N is the biologically active moiety D². Preferred identities for each of n, m, p, q, V, X, Y and Q are as set out above in relation to each of Formulae (I), (I′), (I″), (IIa), (IIa′), (IIb), (IIb′), (Ic) and (IIc′). When L″ is a bond and the biologically active moiety is released by hydrolysis of the C═N bond in vivo, the biologically active molecule is B—ONH₂.

In another aspect, the oxime bond in Formula (IE) or (Ie) may further be reduced. Reduction of an oxime bond may be effected by any known technique in the art. Preferably, in this aspect, the polymer comprises a repeat unit of Formula (IE′), e.g. a repeat unit of Formula (Ie′):

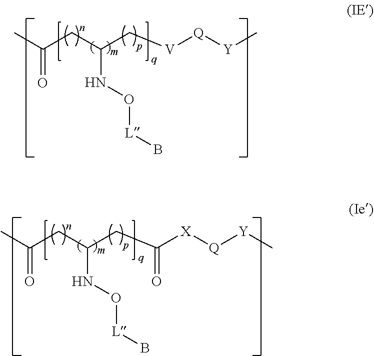

wherein all of the variables are as defined above in relation to Formula (IE) or (Ie).

Especially preferred polymers comprise a repeat unit of Formula (IE), more preferably a repeat unit of Formula (Ie).

Structure of Polymer-Antibody Linker Moieties

This section sets out the possible structural features of the linker moiety present in the antibody-drug conjugates of the invention.

The linker moiety in the antibody-drug conjugates of the present invention may derive from any suitable compound which has at least two separate reactive functional groups: one functional group which reacts with the polymer to form a covalent bond, and a further functional group which reacts with the antibody to form a covalent bond. The antibody-drug linker moiety may be the same or different to any linker group used to attach the polymer backbone to the biologically active moiety (when such a linker group is present). Preferably, the antibody-drug linker moiety is different to the linker group used to attach the polymer backbone to the biologically active moiety.

Typically, the polymer-antibody linker is covalently bound to the polymer through the carbon atom of the -CD¹- moiety in the repeat unit of Formula (I′), (I) or (I″), or the Y group in the repeat unit of Formula (I′), (I) or (I″). Typically, the polymer-antibody linker is covalently bound to the polymer at one of the polymer termini. Alternatively, the polymer-antibody linker is covalently bound to the polymer via condensation with a keto group which is distal from the polymer termini. Preferably, however, the polymer-antibody linker is not covalently bound to the polymer via condensation with a keto group which is distal from the polymer termini.

Typically, the polymer-antibody linker is covalently bound to the antibody through a reactive amino acid side chain of the antibody, e.g. the thiol group of a cysteine residue, the amino group of a lysine residue, the carboxylic acid group of a glutamic acid residue or an aspartic acid residue, the selenol group of a selenocysteine residue, or through the N-terminus of the backbone of one of the polypeptides in the antibody, or through a hydroxyl group of an oligosaccharide present in the fragment crystallisable (Fc) region of the antibody, or through aldehyde or hydroxylamine groups of glycans or non-natural residues, or through alkyne or azide groups of glycans or non-natural residues.

The polymer and the antibody may independently be covalently bound to the same atom of the linker moiety or they may be independently covalently bound to different atoms of the linker moiety. Preferably, the polymer and the antibody are independently covalently bound to different atoms of the linker moiety.

Suitable linker moieties for use in antibody-drug conjugates of the present invention include, but are not limited to, linkers derived from thiols, maleimide, monobromomaleimide, maleimide analogues, vinyl sulfones, bis(sulfone)s, allenamides, vinyl-pyridines, dehydroalanine, alkenes, perfluoroaromatic molecules, sulfone reagents that are Julia-Kocienski like, N-hydroxysuccinamide-ester activated carboxylate species, aldehydes, ketones, hydroxylamines, alkynes and azides.

Thus, reaction of thiols, maleimide, monobromomaleimide, maleimide analogues, vinyl sulfones, bis(sulfone)s, allenamides, vinyl-pyridines, dehydroalanine, alkenes, perfluoroaromatic species, sulfone reagents that are Julia-Kocienski like, N-hydroxysuccinamide-ester activated carboxylate species, aldehydes, ketones, hydroxylamines, alkynes and azides with both (a) the polymer backbone and (b) the antibody results in a suitable linker group L. Bis(sulfones) act in this context as (bis-alkylating) reagents. Linkers can be derived from alkenes by e.g. a light-initiated thiol-ene reaction. Thus, a thiol group on an antibody can react with alkene functionality to generate a covalent link. Reaction with dehydroalanine may occur e.g. by Michael addition-elimination with a thiol group on an antibody. N-hydroxysuccinamide-ester activated carboxylate species may react with lysine groups in an antibody. Ketones, aldehydes and/or hydroxylamines may be conjugated to a glycan-modified antibody or non-natural residue via oxime bond formation or by hydrazino-Pictet-Spengler (HIPS) ligation. Alkynes and azides may be conjugated to a glycan-modified antibody or non-natural residue via click chemistry (azide-alkyne cycloaddition).

Structure of Antibody-Drug Conjugates

Most preferably, the antibody-drug conjugate of the present invention has Formula (III) or (IV):

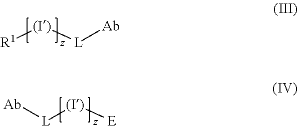

wherein:
(I′) is a repeat unit of the Formula (I′), e.g. a repeat unit of Formula (I) or Formula (I″), as defined above;
Ab is an antibody or antigen-binding fragment thereof as defined above;
L is a polymer-antibody linker as defined above;
R¹ is selected from OH, OR′, SH, SR′, NH₂, NHR′ and NR′₂;
E is selected from H and R′;
R′ is as defined above; and
z is an integer from 2 to 50.

Thus, typically, the antibody-drug conjugate of the present invention has Formula (III′), or (IV′), e.g. Formula (IIIa), (IIIb), (IVa) or (IVb):

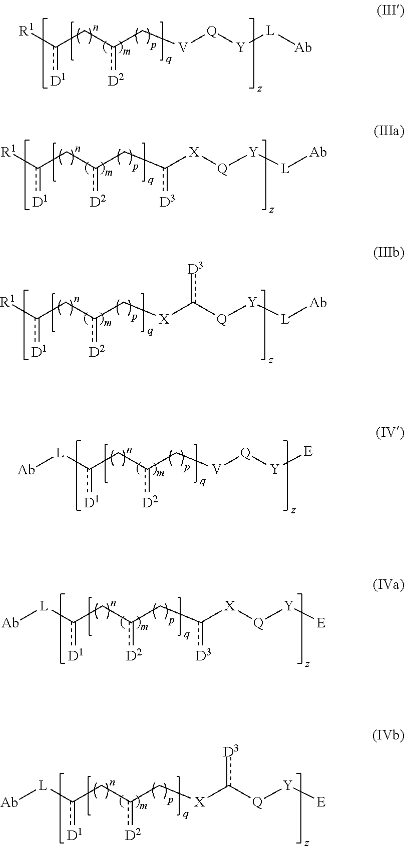

wherein:
Ab is an antibody or antigen-binding fragment thereof as defined above;
L is a polymer-antibody linker as defined above;
R¹ is selected from OH, OR′, SH, SR′, NH₂, NHR′ and NR′₂;
E is H or R′;
each n, m, p, q, V, X, Q, Y, D¹, D², D³ and R′ are as defined above; and
z is an integer from 2 to 50.

Preferably, z is an integer from 2 to 30, more preferably from 2 to 20, even more preferably from 2 to 15, and most preferably from 2 to 12.

The antibody-drug conjugates of the invention may further comprise a second targeting agent. Preferably the targeting agent is covalently bound to the polymer. Suitable targeting agents include biomolecules such as peptide, protein, peptide mimetics, antibodies, antigens, DNA, mRNA, small interfering RNA, small hairpin RNA, microRNA, PNA, foldamers, carbohydrates, carbohydrate derivatives, non-Lipinski molecules, synthetic peptide and synthetic oligonucleotides.

The polymer in an antibody-drug conjugate of the present invention typically has a weight average molecular weight of 500 to 500 000 Da, more preferably 1000 to 200 000 Da, and still more preferably 1500 to 36 000 Da. Preferably, the polymer has a number average molecular weight of 500 to 500 000 Da, more preferably 1000 to 200 000 Da, still more preferably 1500 to 25 000 Da and yet more preferably 2000 to 20 000 Da. Preferably, the polymer has a polydispersity of 1 to 5, more preferably 1.05 to 4.8, still more preferably 1.1 to 2.4 and yet more preferably 1.1 to 1.5.

The biologically active moiety present in the antibody-drug conjugates of the present invention preferably has a molecular weight of 32 to 100 000 Da. The biologically active moiety may be a small molecule drug which may be a small organic molecule, i.e. non-polymeric, or polymeric. Preferably the antibody-drug conjugate of the present invention comprises 0.5 to 90 wt %, more preferably 0.75 to 70 wt %, still more preferably 1 to 60 wt %, yet more preferably 1.5 to 50 wt %, still more preferably 1.75 to 25 wt %, and most preferably 2 to 10 wt % biologically active moiety, based on the weight of the dry antibody-drug conjugate. A key advantage of the antibody-drug conjugates of the present invention is that relatively high amounts of biologically active molecule can be incorporated into the polymer. Further, multiple polymers may bind to a single antibody. These factors, in turn, mean that high biologically active molecule loadings may be achieved. Typically, the drug-to-antibody ratio (DAR) is 4:1 or greater, preferably 5:1 or greater, more preferably 8:1 or greater, yet more preferably 10:1 or greater, still more preferably 12:1 or greater, even more preferably 15:1 or greater, and most preferably 16:1 or greater, for example 20:1 or greater.

Each biologically active moiety B¹, B² and/or B³ in the antibody-drug conjugates of the present invention may be the same. However, preferably, the antibody-drug conjugate of the invention contains at least two different biologically active moieties, for example 2, 3 or 4 different biologically active moieties.

Preferred biologically active moieties present in the antibody-drug conjugates of the present invention are drugs selected from anti-infective, antibiotics, antibacterial, antimicrobial, anti-inflammatory, analgesic, antihypertensive, antifungal, anti-tubercular, antiviral, anticancer, antiplatelet, antimalarial, anticonvulsant, cardio protective, antihelmintic, antiprotozoal, anti-trypanosomal, antischistosomiasis, antineoplastic, antiglaucoma, tranquilizers, hypnotics, anticonvulsants, antiparkinson, antidepressant, antihistaminic, antidiabetic or antiallurgics.

Particularly preferred biologically active moieties present in the antibody-drug conjugates of the present invention are selected from auristatins (e.g. monomethyl auristatin E (MMAE) and MMAF), dolastatins, maytansinoids (e.g. DM1 and DM4), tubulysins, calicheamicins, duocarmycins, benzodiazepines, camptothecin, camptothecin analogues, amatoxin, doxorubicin, and α-amanitin.

Typically, the antibody-drug conjugates of the present invention have a solubility in water of at least 10 mg/mL, preferably at least 30 mg/mL, more preferably at least 50 mg/mL, still more preferably at least 75 mg/mL, and most preferably at least 100 mg/mL.

The present invention also provides an antibody-drug conjugate as described herein, wherein release of the biologically active moiety from the polymer is pH sensitive and is dependent upon the nature of the bond between said biologically active moiety and the repeat unit of the polymer or the linker group to which it is covalently bound.

Alternatively, the antibody may be replaced by an alternative form of targeting agent. Thus, the present invention also provides a targeting agent-drug conjugate comprising:
(i) a targeting agent;
(ii) a polymer comprising a repeat unit of Formula (I′):

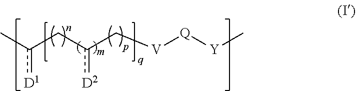

wherein:
each n and each p is independently 0 or an integer between 1 and 6;
each m is independently 0 or an integer between 1 and 4, and preferably at or least one m is 1; V is

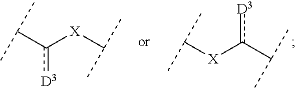

-------- is a bond which may be absent or present;
each D¹ is independently O or L¹-B¹;
each D² is independently O or L²-B²
each D³ is independently O or L³-B³
L¹ is a linker group or a bond, L² is a linker group or a bond, L³ is a linker group or a bond, and each B¹, B² and B³ is a biologically active moiety;
provided that at least one D¹, D² or D³ group within the polymer is not O, and further provided that when D¹, D² or D³ is O, there is a double bond between the O atom and the carbon atom to which it is attached; each q is an integer between 1 and 8;
X and Y are independently selected from O, NH, NR′ and S;
R′ is C₁₋₂₀ hydrocarbyl;
Q is selected from —CH₂(NMe(C═O)CH₂)ₒ—, -T¹O(CH₂CH₂O)ₛT²- and -T¹O(CH₂CH₂CH₂O)ₛT²-, wherein T¹ is selected from a divalent methylene, ethylene, propylene or butylene radical, and T² is selected from a divalent methylene, ethylene, propylene or butylene radical;
is an integer from 0 to 100; and
s is an integer from 0 to 150; and
(iii) a polymer-targeting agent linker which is covalently bonded to both the targeting agent and the polymer.

Preferably, the polymer comprises a repeat unit of Formula (I):

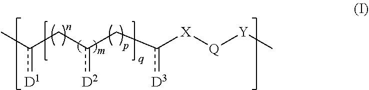

wherein the variables X, Y, D¹, D², D³, n, m and p are as set out above, and Q is selected from -T¹O(CH₂CH₂O)ₛT²- and -T¹O(CH₂CH₂CH₂O)ₛT²-.

Alternatively, the polymer comprises a repeat unit of Formula (I″):

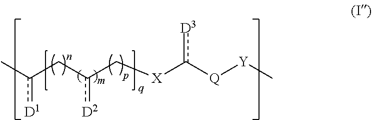

wherein the variables X, Y, Q, D¹, D², D³, n, m and p are as set out above. Preferably, in the repeat unit of Formula (I″), Q is —CH₂(NMe(C═O)CH₂)ₒ—. More preferably, Q is —CH—₂(NMe(C═O)CH₂)ₒ— and Y is —NMe.

The targeting agent is covalently bound to the polymer. Suitable targeting agents include biomolecules such as peptides, proteins, peptide mimetics, antibodies, antigens, DNA, mRNA, small interfering RNA, small hairpin RNA, microRNA, PNA, foldamers, carbohydrates, carbohydrate derivatives, non-Lipinski molecules, synthetic peptides and synthetic oligonucleotides.

The polymer-targeting agent linker may assume any of the same structures as the polymer-antibody linker that is defined above.

Most preferably, the targeting agent-drug conjugate of the present invention has Formula (V) or (VI):

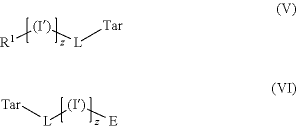

wherein:
(I′) is a repeat unit of the Formula (I′), e.g. a repeat unit of Formula (I) or Formula
(I″), as defined above;
Tar is a targeting agent as defined above;
L is a polymer-targeting agent linker as defined above;
R¹ is selected from OH, OR′, SH, SR′, NH₂, NHR′ and NR′₂;
E is selected from H and R′;
R′ is as defined above; and
z is an integer from 2 to 50.

Thus, typically, the targeting agent-drug conjugate of the present invention has Formula (V′) or (VI′), e.g. Formula (Va), (Vb), (Via) or (VIb):

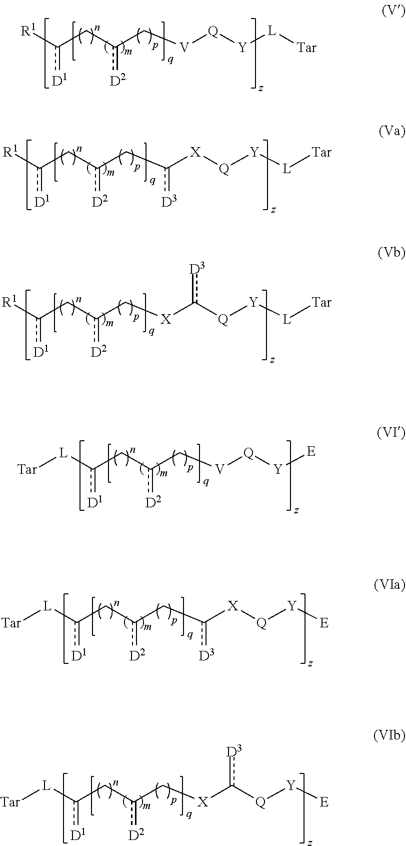

wherein:
Tar is a targeting agent as defined above;
L is a polymer-targeting agent linker as defined above;
R¹ is selected from OH, OR′, SH, SR′, NH₂, NHR′ and NR′₂;
E is H or R′;
each n, m, p, q, X, Q, Y, D¹, D², D³ and R′ are as defined above; and
z is an integer from 2 to 50.

Preferably, z is an integer from 2 to 30, more preferably from 2 to 20, even more preferably from 2 to 15, and most preferably from 2 to 12. The polymer in a targeting agent-drug conjugate of the present invention typically has a weight average molecular weight of 500 to 500 000 Da, more preferably 1000 to 200 000 Da, and still more preferably 1500 to 36 000 Da. Preferably, the polymer has a number average molecular weight of 500 to 500 000 Da, more preferably 1000 to 200 000 Da, still more preferably 1500 to 25 000 Da and yet more preferably 2000 to 20 000 Da. Preferably, the polymer has a polydispersity of 1 to 5, more preferably 1.05 to 4.8, still more preferably 1.1 to 2.4 and yet more preferably 1.1 to 1.5.

The biologically active moiety present in the targeting agent-drug conjugates of the present invention preferably has a molecular weight of 32 to 100 000 Da. The biologically active moiety may be a small molecule drug which may be a small organic molecule, i.e. non-polymeric, or polymeric. Preferably the targeting agent-drug conjugate of the present invention comprises 0.5 to 90 wt %, more preferably 0.75 to 70 wt %, still more preferably 1 to 60 wt %, yet more preferably 1.5 to 50 wt %, even more preferably 1.75 to 25 wt %, and most preferably 2 to 10 wt % biologically active moiety, based on the weight of the dry antibody-drug conjugate. A key advantage of the targeting agent-drug conjugates of the present invention is that relatively high amounts of biologically active molecule can be incorporated into the polymer. Further, multiple polymers may bind to a single targeting agent. These factors, in turn, mean that high biologically active molecule loadings may be achieved. Typically, the drug-to-targeting agent ratio is 4:1 or greater, preferably 5:1 or greater, more preferably 8:1 or greater, yet more preferably 10:1 or greater, still more preferably 12:1 or greater, even more preferably 15:1 or greater, and most preferably 16:1 or greater, for example 20:1 or greater.

Each biologically active moiety B¹, B² and/or B³ in the targeting agent-drug conjugates of the present invention may be the same. Alternatively, the targeting agent-drug conjugate of the invention contains at least two different biologically active moieties, for example 2, 3 or 4 different biologically active moieties. Preferred biologically active moieties present in the targeting-drug conjugates of the present invention are as described above in relation to antibody-drug conjugates.

Typically, the targeting agent-drug conjugates of the present invention have a solubility in water of at least 30 mg/mL, preferably at least 50 mg/mL, more preferably at least 75 mg/mL, and most preferably at least 100 mg/mL.

Methods for Manufacture of Antibody-Drug Conjugates

The present invention also relates to a method of producing an antibody-drug conjugate according to the invention.

Typically, such a method comprises the steps of:
(a) reacting a compound of Formula (IIa):

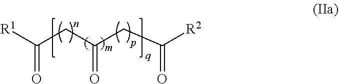

with a compound of Formula (IIb):

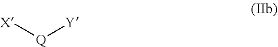

wherein R¹, R², n, m, p, q, X′, Y′ and Q are as defined above;
(b) reacting the product of step (a) with a polymer-antibody linker;
(c) optionally, reacting the product of step (b) with a compound of formula HL¹-LG, HL²-LG or HL³-LG, wherein L¹, L², L³ and LG are as defined above;
(d) reacting the product of step (c) with a biologically active molecule, or if step (c) is not performed, reacting the product of step (b) with a biologically active molecule; and
(e) reacting the product of step (d) with an antibody or antigen-binding fragment thereof.

Alternatively, the method comprises the steps of:
(a) reacting a compound of Formula (IIa):

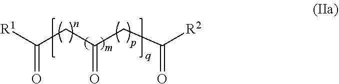

with a compound of Formula (IIb):

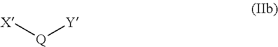

wherein R¹, R², n, m, p, q, X′, Y′ and Q are as defined above;
(b) optionally, reacting the product of step (b) with a compound of formula HL¹-LG, HL²-LG or HL³-LG, wherein L¹, L², L³ and LG are as defined above;
(c) reacting the product of step (b) with a biologically active molecule, or if step (c) is not performed, reacting the product of step (b) with a biologically active molecule;
(d) reacting the product of step (c) with a polymer-antibody linker; and
(e) reacting the product of step (d) with an antibody or antigen-binding fragment thereof.
Alternatively, the method comprises the steps of:
(a) reacting a compound of Formula (IIa):

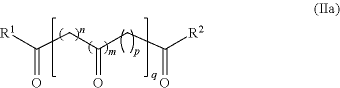

with a compound of Formula (IIb):

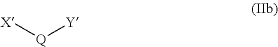

and a biologically active molecule,
and optionally, a compound of formula HL¹-LG, HL²-LG or HL³-LG, wherein R¹, R², n, m, p, q, X′, Y′, Q, L¹, L², L³ and LG are as defined above;
(b) reacting the product of step (a) with a polymer-antibody linker; and
(c) reacting the product of step (b) with an antibody or antigen-binding fragment thereof.
Alternatively, the method comprises the steps of:
(a) reacting an antibody or antigen-binding fragment thereof with a polymer-antibody linker;
(b) separately, reacting a compound of Formula (IIa):

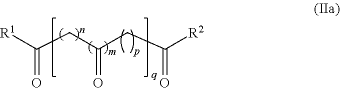

with a compound of Formula (IIb):

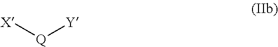

wherein R¹, R², n, m, p, q, X′, Y′ and Q are as defined above;
(c) reacting the product of step (a) with the product of step (b);
(d) optionally, reacting the product of step (c) with a compound of formula HL¹-LG, HL²-LG or HL³-LG, wherein L¹, L², L³ and LG are as defined above; and
(e) reacting the product of step (d) with a biologically active molecule, or if step (d) is not performed, reacting the product of step (c) with a biologically active molecule.
Alternatively, the method comprises the steps of:
(a) reacting an antibody or antigen-binding fragment thereof with a polymer-antibody linker;
(b) separately, reacting a compound of Formula (IIa):

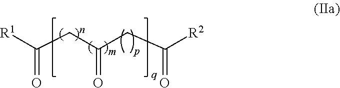

with a compound of Formula (IIb):

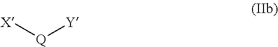

wherein R¹, R², n, m, p, q, X′, Y′ and Q are as defined above;
(c) optionally, reacting the product of step (b) with a compound of formula HL¹-LG, HL²-LG or HL³-LG, wherein L¹, L², L³ and LG are as defined above;
(d) reacting the product of step (c) with a biologically active molecule, or if step (c) is not performed, reacting the product of step (b) with a biologically active molecule; and
(d) reacting the product of step (a) with the product of step (d).

Alternatively, such a method comprises the steps of:
(a) reacting a compound of Formula (IIa′):

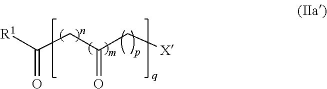

with a compound of Formula (IIb′):

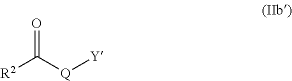

wherein R¹, R², n, m, p, q, X′, Y′ and Q are as defined above;
(b) reacting the product of step (a) with a polymer-antibody linker;
(c) optionally, reacting the product of step (b) with a compound of formula HL¹-LG, HL²-LG or HL³-LG, wherein L¹, L², L³ and LG are as defined above;
(d) reacting the product of step (c) with a biologically active molecule, or if step (c) is not performed, reacting the product of step (b) with a biologically active molecule; and
(e) reacting the product of step (d) with an antibody or antigen-binding fragment thereof.

Alternatively, the method comprises the steps of:
(a) reacting a compound of Formula (IIa′):

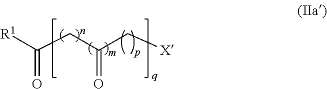

with a compound of Formula (IIb′):

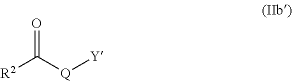

wherein R¹, R², n, m, p, q, X′, Y′ and Q are as defined above;
(b) optionally, reacting the product of step (b) with a compound of formula HL¹-LG, HL²-LG or HL³-LG, wherein L¹, L², L³ and LG are as defined above;
(c) reacting the product of step (b) with a biologically active molecule, or if step (c) is not performed, reacting the product of step (b) with a biologically active molecule;
(d) reacting the product of step (c) with a polymer-antibody linker; and
(e) reacting the product of step (d) with an antibody or antigen-binding fragment thereof.
Alternatively, the method comprises the steps of:
(a) reacting a compound of Formula (IIa′):

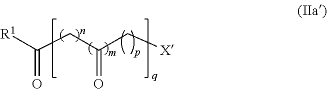

with a compound of Formula (IIb′):

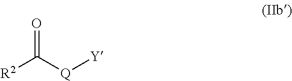

and a biologically active molecule,
and optionally, a compound of formula HL¹-LG, HL²-LG or HL³-LG, wherein R¹, R², n, m, p, q, X′, Y′, Q, L¹, L², L³ and LG are as defined above;
(b) reacting the product of step (a) with a polymer-antibody linker; and
(c) reacting the product of step (b) with an antibody or antigen-binding fragment thereof.
Alternatively, the method comprises the steps of:
(a) reacting an antibody or antigen-binding fragment thereof with a polymer-antibody linker;
(b) separately, reacting a compound of Formula (IIa′):

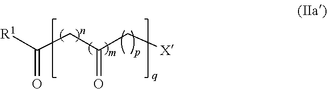

with a compound of Formula (IIb′):

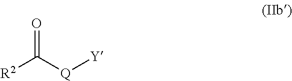

wherein R¹, R², n, m, p, q, X′, Y′ and Q are as defined above;
(c) reacting the product of step (a) with the product of step (b);
(d) optionally, reacting the product of step (c) with a compound of formula HL¹-LG, HL²-LG or HL³-LG, wherein L¹, L², L³ and LG are as defined above; and
(e) reacting the product of step (d) with a biologically active molecule, or if step (d) is not performed, reacting the product of step (c) with a biologically active molecule.
Alternatively, the method comprises the steps of:
(a) reacting an antibody or antigen-binding fragment thereof with a polymer-antibody linker;
(b) separately, reacting a compound of Formula (IIa′):

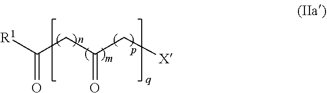

with a compound of Formula (IIb′):

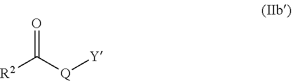

wherein R¹, R², n, m, p, q, X′, Y′ and Q are as defined above;
(c) optionally, reacting the product of step (b) with a compound of formula HL¹-LG, HL²-LG or HL³-LG, wherein L¹, L², L³ and LG are as defined above;
(d) reacting the product of step (c) with a biologically active molecule, or if step (c) is not performed, reacting the product of step (b) with a biologically active molecule; and
(d) reacting the product of step (a) with the product of step (d).

In any of the above embodiments, when the method involves two sequential steps in which (i) a compound of formula HL¹-LG, HL²-LG or HL³-LG is added to an intermediate product, followed by (ii) a step in which a biologically active molecule is added, steps (i) and (ii) of said method may also be amended such that the compound of formula HL¹-LG, HL²-LG or HL³-LG is first reacted with a biologically active molecule to form a compound of formula HL¹-B¹, HL²-B² or HL³-B³, prior to subsequent reaction of the resultant compound HL¹-B¹, HL²-B² or HL³-B³ with said intermediate product.

A particularly preferred method comprises the steps of:
(a) reacting a compound of Formula (IIa):

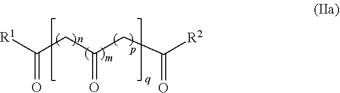

with a compound of Formula (IIb):

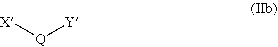

wherein R¹, R², n, m, p, q, X′, Y′ and Q are as defined above;
(b) reacting the product of step (a) with a polymer-antibody linker;
(c) reacting a compound of formula HL¹-LG, HL²-LG or HL³-LG, wherein L¹, L², L³ and LG are as defined above, with a biologically active molecule;
(d) reacting the product of step (b) with the product of step (c); and
(e) reacting the product of step (d) with an antibody or antigen-binding fragment thereof.

In preferred methods of the invention, the biologically active molecule is as defined herein or a protected version of a biologically active molecule as defined herein. Conventional protecting group strategies, as are well known in the art, may be employed during the polymerisation, functionalization and conjugation reactions. In further preferred methods of the invention, the antibody is as defined herein. In yet further preferred methods of the invention, the polymer-antibody linker moiety is as defined herein.

The polymerisation step in the methods of the invention is preferably carried out enzymatically or by polycondensation, free radical chain growth polymerisation or ring-opening polymerisation, most preferably enzymatically.

Pharmaceutical Compositions

The antibody-drug conjugates of the present invention may be incorporated into pharmaceutical compositions. Thus, the present invention provides a pharmaceutical composition comprising an antibody-drug conjugate as defined herein, and one or more pharmaceutically acceptable carriers, diluents or excipients. Pharmaceutical compositions may be prepared in any conventional manner. A pharmaceutical composition may comprise one or more different antibody-drug conjugates as described herein. Suitable carriers, diluents and excipients are well known in the art.

Pharmaceutical compositions of the invention may be administered to a patient by any one or more of the following routes: oral, systemic (e.g. transdermal, intranasal, transmucosal or by suppository), or parenteral (e.g. intramuscular, intravenous or subcutaneous). Compositions of the invention can take the form of tablets, pills, capsules, semisolids, powders, sustained release formulations, solutions, suspensions, elixirs, aerosols, transdermal patches, bioadhesive films, or any other appropriate compositions. The choice of formulation depends on various factors such as the mode of drug administration (e.g. for oral administration, formulations in the form of tablets, pills or capsules are preferred) and the bioavailability of the drug substance.

The pharmaceutical compositions of the invention may additionally include common pharmaceutical excipients such as lubricating agents, thickening agents, wetting agents, emulsifying agents, suspending agents, preserving agents, fillers, binders, preservatives and adsorption enhancers, e.g. surface penetrating agents. Solubilizing and/or stabilizing agents may also be used, e.g. cyclodextrins (CD). A person skilled in the art will be able to select suitable excipients based on their purpose. Common excipients that may be used in the pharmaceutical products herein described are listed in various handbooks (e.g. D. E. Bugay and W. P. Findlay (Eds) Pharmaceutical excipients (Marcel Dekker, New York, 999), E-M Hoepfner, A. Reng and P. C. Schmidt (Eds) Fiedler Encyclopedia of Excipients for Pharmaceuticals, Cosmetics and Related Areas (Edition Cantor, Munich, 2002) and H. P. Fielder (Ed) Lexikon der Hilfsstoffe fur Pharmazie, Kosmetik und angrenzende Gebiete (Edition Cantor Aulendorf, 1989)).

The pharmaceutical compositions of the invention may be formulated so as to provide quick, sustained or delayed release of the antibody-drug conjugate after administration to the patient by employing procedures well known in the art. The concentration of the antibody-drug conjugates in the pharmaceutical compositions depends upon numerous factors including the nature of the polymer, the drug loading on the polymer, the identity of the antibody, the composition, the mode of administration, the condition to be treated or diagnosed, and the subject to which it is administered and may be varied or adjusted according to choice by techniques well-known to a person of skill in the art.

Medical Uses of the Antibody-Drug Conjugates

The antibody-drug conjugates and pharmaceutical compositions described herein are useful in medical applications. Thus, the present invention provides an antibody-drug conjugate as described herein for use in the treatment of a disease or condition in a patient in need thereof. Typically, the antibody-drug conjugates and pharmaceutical compositions described herein are for use in the treatment of a disease selected from inflammatory diseases (e.g. inflammatory bowel disease, rheumatoid arthritis and artherosclerosis), metabolic disorders (e.g. diabetes, insulin resistance, obesity), cancer, bacterial infections (e.g. Tuberculosis, pneumonia, endocarditis, septicaemia, salmonellosis, typhoid fever, cystic fibrosis, chronic obstructive pulmonary diseases), viral infections, cardiovascular diseases, neurodegenerative diseases, neurological disorders, behavioural and mental disorders, blood diseases, chromosome disorders, congenital and genetic diseases, connective tissue diseases, digestive diseases, ear, nose, and throat diseases, endocrine diseases, environmental diseases, eye diseases, female reproductive diseases, fungal infections, heart diseases, hereditary cancer syndromes, immune system diseases, kidney and urinary diseases, lung diseases, male reproductive diseases, mouth diseases, musculoskeletal diseases, myelodysplastic syndromes, nervous system diseases, newborn screening, nutritional diseases, parasitic diseases, rare cancers, and skin diseases.

In general, antibody-drug conjugates of the present invention are administered to a human patient so as to deliver to the patient a therapeutically effective amount of the biologically active molecule contained therein.

As used herein, the term “therapeutically effective amount” refers to an amount of the biologically active molecule which is sufficient to reduce or ameliorate the severity, duration, progression, or onset of a disorder being treated, prevent the advancement of a disorder being treated, cause the regression of, prevent the recurrence, development, onset or progression of a symptom associated with a disorder being treated, or enhance or improve the prophylactic or therapeutic effect(s) of another therapy. The precise amount of biologically active molecule administered to a patient will depend on the type and severity of the disease or condition and on the characteristics of the patient, such as general health, age, sex, body weight and tolerance to drugs. It will also depend on the degree, severity and type of the disorder being treated. The skilled artisan will be able to determine appropriate dosages depending on these and other factors.

As used herein, the terms “treat”, “treatment” and “treating” refer to the reduction or amelioration of the progression, severity and/or duration of a disorder being treated, or the amelioration of one or more symptoms (preferably, one or more discernible symptoms) of a disorder being treated resulting from the administration of a film according to the invention to a patient.

The present invention also provides a method of treating a disease or condition as described herein in a human patient, wherein said method comprises administration of at least one antibody-drug conjugate as described herein to a patient in need thereof.

The present invention also provides the use of an antibody-drug conjugate as described herein for the manufacture of a medicament for the treatment of a disease or condition as described herein in a human patient.

Any antibody-drug conjugate or antibody-drug conjugates of the present invention may also be used in combination with one or more other drugs or pharmaceutical compositions in the treatment of disease or conditions for which the ADCs of the present invention and/or the other drugs or pharmaceutical compositions may have utility.

The one or more other drugs or pharmaceutical compositions may be administered to the patient by any one or more of the following routes: oral, systemic (e.g. transdermal, intranasal, transmucosal or by suppository), or parenteral (e.g. intramuscular, intravenous or subcutaneous). Compositions of the one or more other drugs or pharmaceutical compositions can take the form of tablets, pills, capsules, semisolids, powders, sustained release formulations, solutions, suspensions, elixirs, aerosols, transdermal patches, bioadhesive films, or any other appropriate compositions. The choice of formulation depends on various factors such as the mode of drug administration (e.g. for oral administration, formulations in the form of tablets, pills or capsules are preferred) and the bioavailability of the drug substance.

The publications, patent publications and other patent documents cited herein are entirely incorporated by reference. Herein, any reference to a term in the singular also encompasses its plural. Where the term “comprising”, “comprise” or “comprises” is used, said term may substituted by “consisting of”, “consist of” or “consists of” respectively, or by “consisting essentially of”, “consist essentially of” or “consists essentially of” respectively. Any reference to a numerical range or single numerical value also includes values that are about that range or single value. Any reference to a polymer having a repeat unit of Formula (I′), (I) or (I″) also encompasses a physiologically acceptable salt thereof unless otherwise indicated. Unless otherwise indicated, any % value is based on the relative weight of the component or components in question.

### EXAMPLES

The following are Examples that illustrate the present invention. However, these Examples are in no way intended to limit the scope of the invention.

### Example 1: Preparation of Polymers

A polymer of Formula (I′) was synthesised and attached to a maleimide group for subsequent conjugation to an antibody via the following synthetic steps.

Step a: Synthesis of Polyamide Backbone

Polyamides were synthesized by enzymatic polycondensation between two building blocks: dimethyl-2-oxoglutarate and poly(ethylene glycol) bis(3-aminopropyl) terminated Mₙ˜1,500 (PEG 1500 diamine) (Scheme 1).

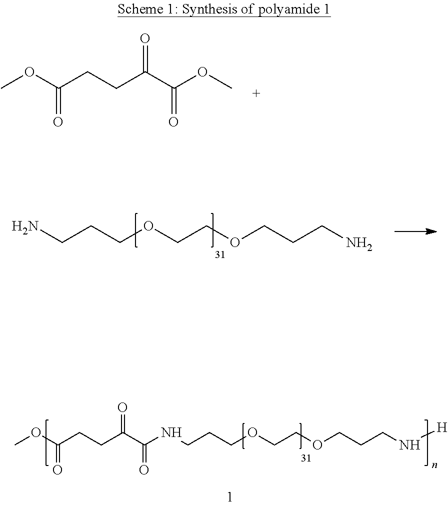

Candida Antarctica Lipase B (CALB) as immobilised enzyme beads (Novozym™ 435, (N435) containing 10 w/w % CALB and 90 w/w % acrylic resin) was dried in a desiccator under vacuum for 2 hours on the day prior to the reaction and kept in the desiccator overnight. It was then added to a round-bottomed flask containing PEG 1500 diamine. Finally, 1.0 equivalent of dimethyl-2-oxoglutarate was added to the round-bottomed flask and a slow flow of nitrogen was kept flushing over the reaction mixture. Overall, the enzyme beads used were equivalent to 10% of the total monomer weight. During the first stage of the reaction, the bulk was heated to 75° C. on a hot plate using an oil bath with magnetic stirring at 200 revolutions per minute (rpm) for 1 hour. Diphenyl ether (300% monomer weight) was then added to reduce viscosity and the temperature was increased to 105° C. while stirring was increased to 300 rpm during the second stage of the reaction. A chemo-resistant diaphragm vacuum pump was connected to the vessel and run continuously. After 24 hours, the reaction was stopped and quenched by addition of chloroform. After leaving time for the polymer product to dissolve, the solution was filtered through cotton fibre to remove enzyme beads. Finally, the filtrate solution was purified by washing with hexane three times yielding a solid precipitate. Polyamide 1 was dried under N₂ and then under vacuum to give a dark brown amorphous solid (yield=55-71% over 4 batches of polymers).

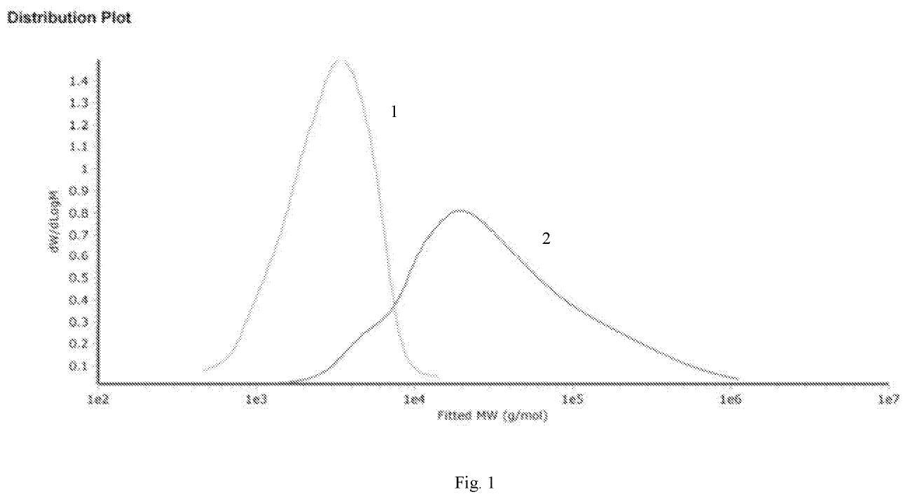

Gel permeation chromatography (GPC) was used to analyse the molecular weight and polydispersity of the polymers. Two analytical PLGel 10 μm MiniMix-B 250×4.6 mm columns equipped with a guard were used for analysis. A 0.3 mL min⁻¹ flow of chloroform was used and the system was calibrated with polystyrene standards. Samples were dissolved in chloroform and filtered through 0.45 μm PTFE syringe filters before injection. (see FIG. 1).

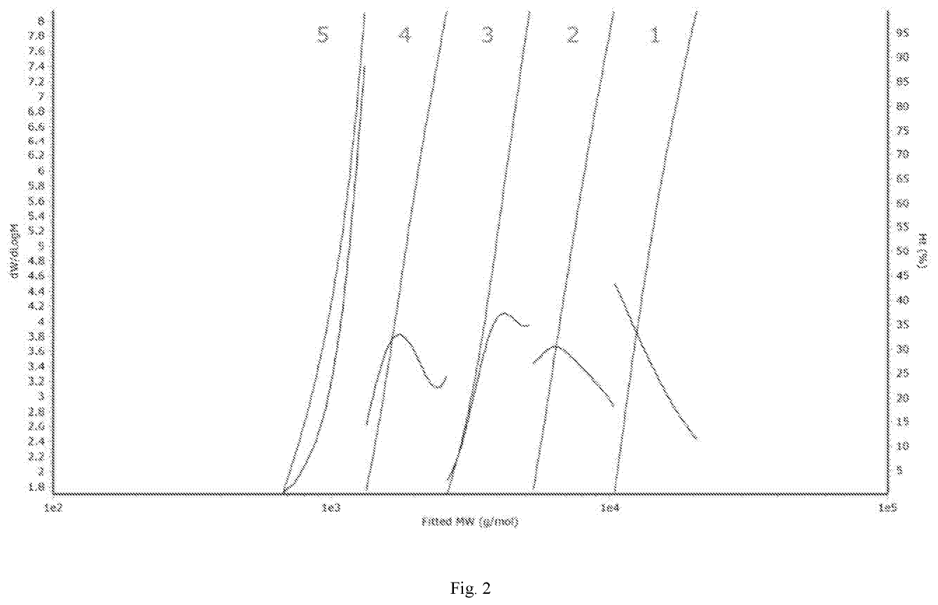

Size exclusion chromatography (SEC) was also used to analyse the molecular weight and polydispersity of polymers. SEC conditions were PL-aquagel-OH 30, 8 μm, 300×7.5 mm column, water as eluant at 0.5 ml min⁻¹ and PEG standards. Samples were dissolved in water and filtered through 0.45p m PES syringe filters before injection. SEC data of polyamide 1 showed a distribution of polymers having between 1 (Mₙ=1 kDa and 1.8 kDa) and 8 (Mₙ=13.9 kDa) repeat units (see FIG. 2).

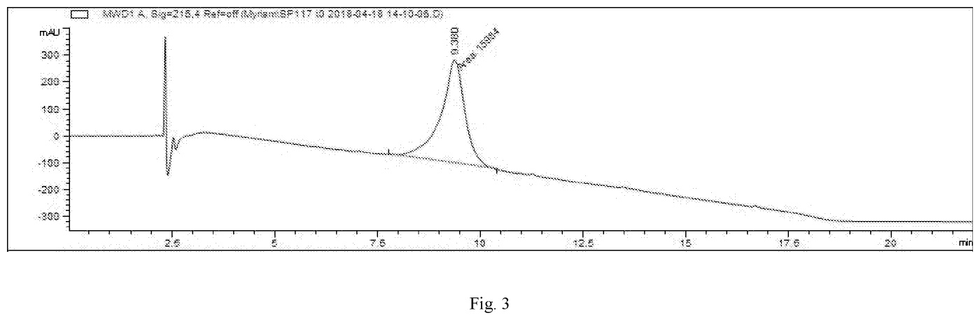

Polyamide 1 was characterized by RP-HPLC (C18 column, H₂O (0.1% TFA) and ACN as an eluent system). The RP-HPLC chromatogram of polyamide 1 is shown in FIG. 3. It displays a peak eluting at RT (retention time)=9.38 minutes (FIG. 3).

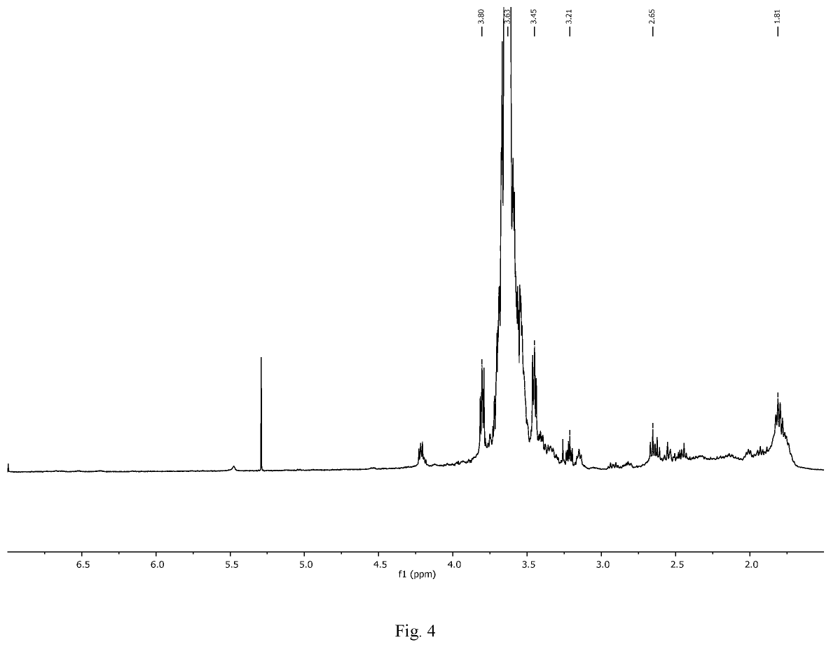

Polyamide 1 was thoroughly dried under vacuum and dissolved in CDCl₃ for NMR analysis (see FIG. 4). The signals are not, however, simple to interpret as the product is a mixture of different chain lengths. Further analysis is required to confirm the signal assignments and provisional assignments are described as follows: ¹H NMR: 3.80 ppm (4 nH, m); 3.63 ppm (124 nH, m); 3.45 (4 nH, m); 3.21 ppm (2 nH, t); 2.65 ppm (2 nH, t); 1.81 ppm (2 nH, m).

Step b: Maleimide Functionalisation of Polyamides

Polyamides 1 obtained after the steps described in the previous section were functionalized with a maleimide group (see Scheme 2).

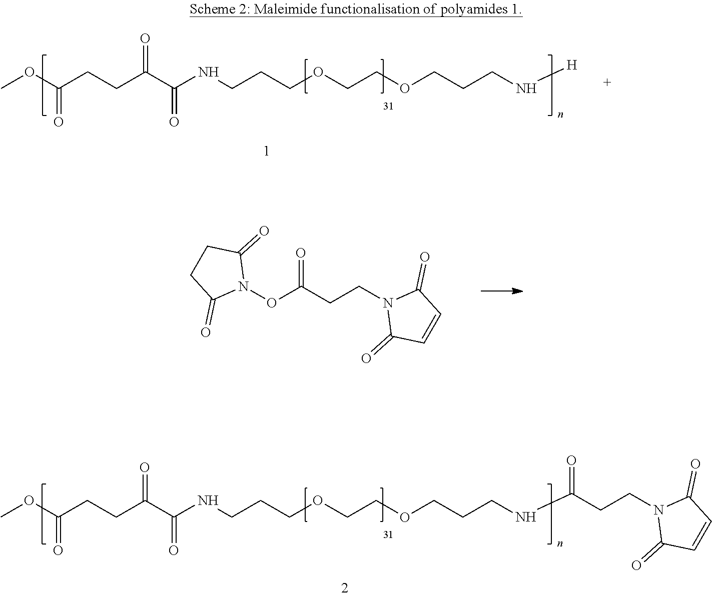

Polyamides 1 were dissolved in dichloromethane at a concentration of 60 mg.ml⁻¹. After leaving the solution stirring to dissolve the polymer, 1.5 equivalents of DIPEA were added to bring the pH of the solution above 9.0. Subsequently, 1.5 equivalents of 3-maleimidopropionic acid N-hydroxysuccinimide (NHS) ester kept and weighed under a bell of nitrogen were added to the reaction mixture, which was left stirring at room temperature for 3 hours. After this time, the contents of the round-bottomed flask were precipitated in at least three times the volume of hexane. The solid precipitate was dissolved in dichloromethane and washed with hexane two more times. Finally, polyamide 2 was dried under nitrogen and then under vacuum. The overall yield of the polyamide synthesis followed by maleimide functionalisation was found to be around 50% on average.

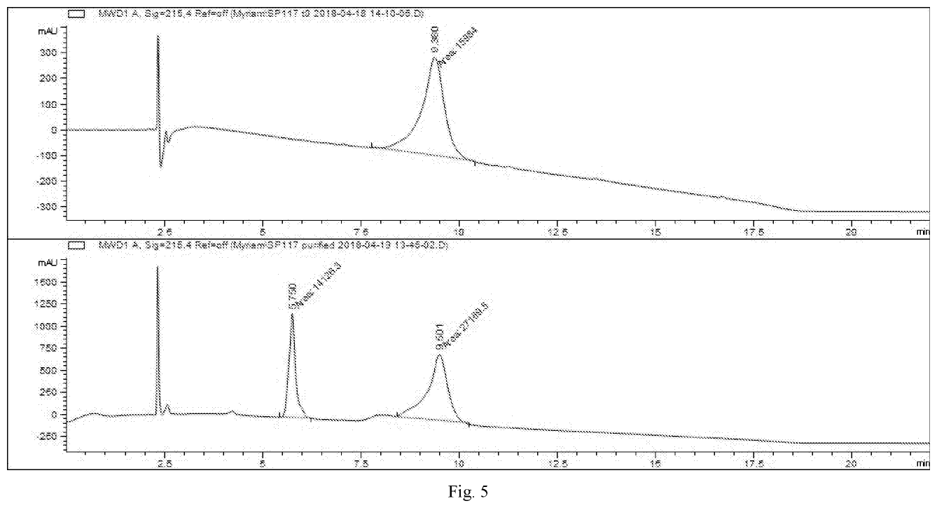

The reaction was monitored by RP-HPLC (see FIG. 5). The polymer peak shifted slightly towards longer retention times with RT=9.5 min compared to 9.38 min at time 0. Given 3-maleimidopropionic acid NHS ester is used in excess, it can be seen in the chromatogram at RT=5.75 min after hexane washes.

Step c: Purification of Maleimide-Functionalised Polyamides

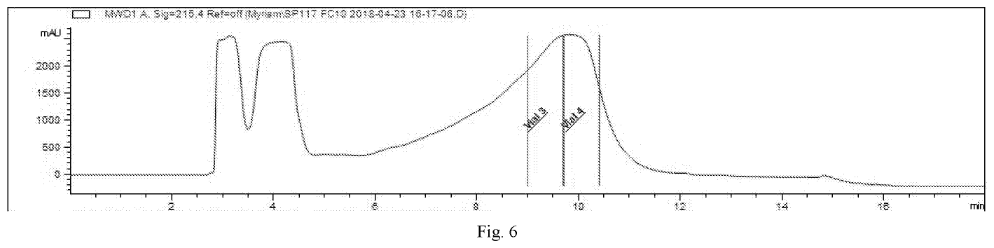

The maleimide functionalised polyamides 2 were purified by semi-preparative RP-HPLC (see FIG. 6). While this does not allow separation of the polymers by molecular weight, this method does enable removal of remaining starting materials. Polymers were dissolved in ACN:H2O (50:50 v/v %) at a concentration of 50 mg mL⁻¹ and centrifuged at 13000 rpm for 10 minutes. The supernatant was collected and injected into a semi-preparative RP-HPLC system using a flow of 4 mL.min⁻¹ with a 30 to 56% in 13 minutes gradient of ACN:H₂O (0.1% TFA). Time-based fractions of the polymer peaks were collected and then freeze-dried to yield RP-HPLC purified products.

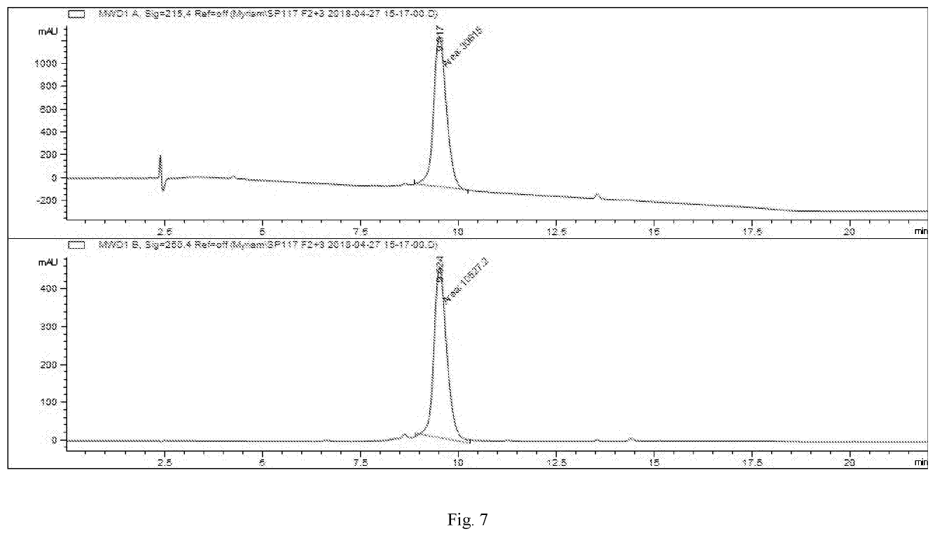

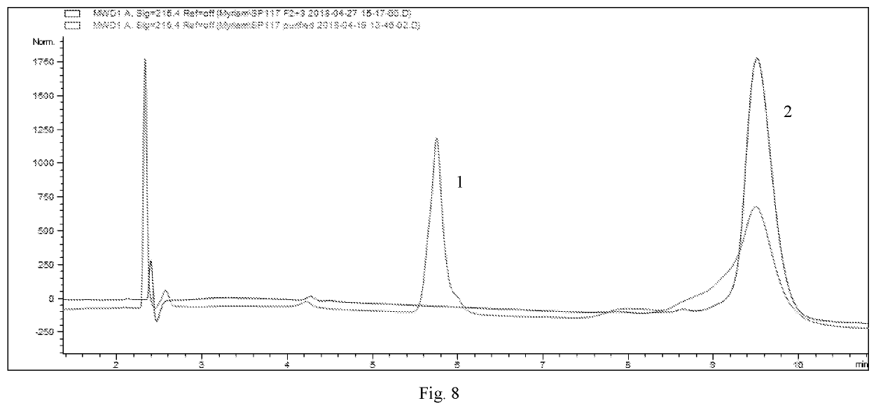

The chromatogram for RP-HPLC purified polymer shows a single peak at RT=9.52 min (see FIG. 7). The polymer peak is also sharper than prior to the purification step (see FIG. 8).

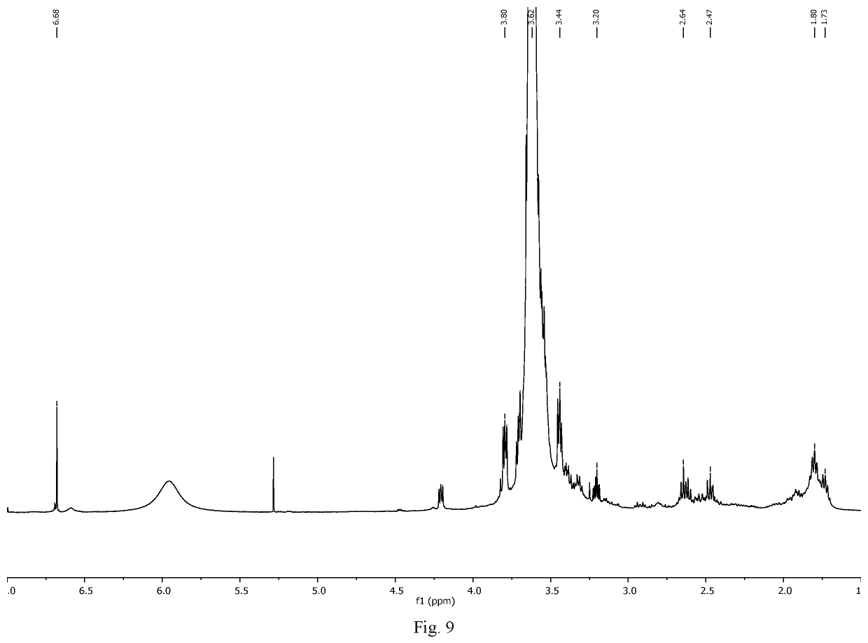

The ¹H NMR spectrum of polyamide 2 after purification by RP-HPLC is provided in FIG. 9. The signals are not, however, simple to interpret as the product is a mixture of different chain lengths. Further analysis is required to confirm the signal assignments and provisional assignments are described as follows: ¹H NMR: 6.68 ppm (2H, s); 3.80 ppm (4 nH, m); 3.62 ppm (124 nH, m); 3.44 (4 nH, m); 3.20 ppm (2 nH, t); 2.64 ppm (2 nH, t); 2.47 ppm (2H, t); 1.80-1.73 ppm (2 nH, m).

The ¹H-NMR spectrum confirms the successful formation of maleimide-functionalized polyamides 2. After purification, a single maleimide peak remains at δ=6.68 ppm, confirming the presence of a maleimide group.

Determining the Water Solubility of Maleimide-Functionalised Polyamides 2

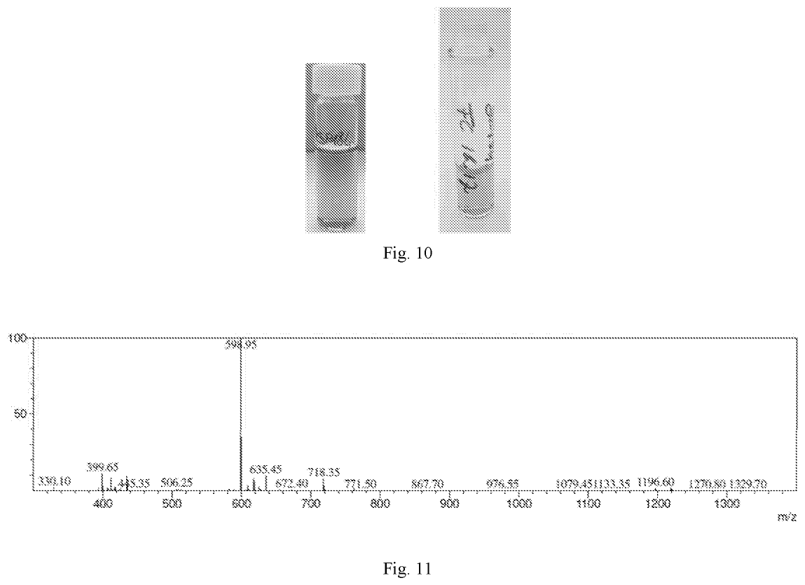

The solubility of polyamides 2 in aqueous solutions was tested. Crude and RP-HPLC purified polyamides 2 were soluble at concentrations ≥50 mg mL⁻¹, as demonstrated by the samples photographed in FIG. 10.

### Example 2: Preparation of MMAE

MMAE was selected as an example drug for conjugation onto the polyamides 2.

MMAE toxin agent was used as a modified MMAE with a cathepsin B sensitive valine-citrulline (Val-Cit) dipeptide separated by the self immolative p-aminobenyloxycarbonyl (PABC) linker. A linker (or cross-linker) was introduced on MMAE to enable the conjugation of MMAE to polyamide 2 via oxime bond formation via the synthetic procedure detailed below.

Step a: Fmoc removalfrom Fmoc-Val-Cit-PABC-MMAE

Fmoc removal from Fmoc-Val-Cit-PABC-MMAE was performed in the presence of diethylamine (Scheme 3).

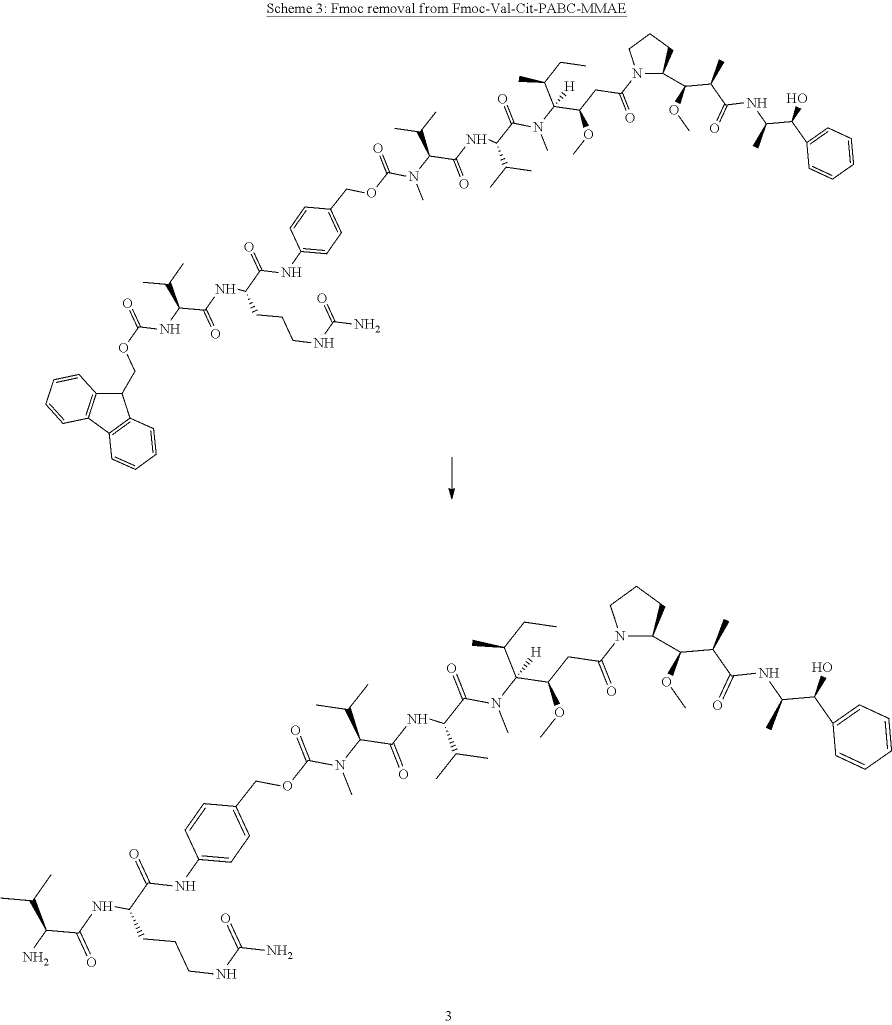 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)OCC2c3ccccc3-c3ccccc32)C(C)C)cc1)C(C)C ; CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](N)C(C)C)cc1)C(C)C]

135 mg of Fmoc-Val-Cit-PABC-MMAE (MMAE) were dissolved in 2 mL anhydrous DMF in a powder box and transferred in a 10 mL round bottom flask (RBF). The RBF was placed under N₂ for a few minutes and 335 μl of diethylamine were then added in two portions. The reaction mixture was left stirring at RT under N₂ for 2.5 hours. The reaction was monitored by normal phase TLC (Silica Gel 60 F₂₅₄). After reaction was stopped, the mixture was concentrated under reduced pressure using an appropriate rotary evaporator system. The yellow residue was washed with diethyl ether three times. The product 3 was recovered as a yellow solid oil.

Step b: Coupling of NHS-Activated Aminoxy-Cross Linker to MMAE

(Boc-aminooxy)acetic N-Hydroxysuccinimide (NHS) ester activated (NHS-activated aminoxy) cross-linker was coupled to MMAE 3 (Scheme 4).

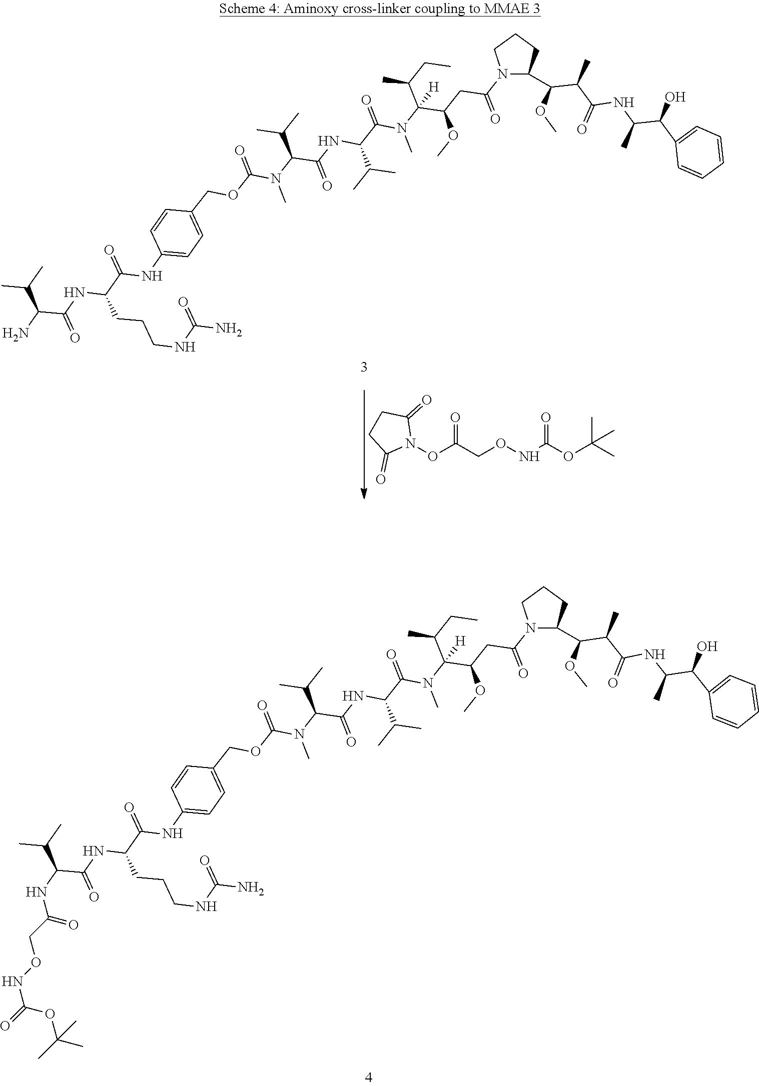 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](N)C(C)C)cc1)C(C)C ; CC(C)(C)OC(=O)NOCC(=O)ON1C(=O)CCC1=O ; CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)CONC(=O)OC(C)(C)C)C(C)C)cc1)C(C)C]

MMAE 3 was dissolved in 2 mL DMF and left stirring under N₂. 21 mg of NHS-activated aminoxy cross linker were weighed in a glass vial and dissolved in 1 mL of DMF. This solution was then added to the reaction vessel. Finally, 20 μL of DIPEA/DMF (50:50 v/v %) were added to the reaction mixture. The reaction was left stirring at RT under N₂ for 18 hours. The reaction was monitored by normal phase TLC. After 18 hours of reaction, the reaction solvent was evaporated under vacuum to obtain a solid product.

The product MMAE 4 was washed with diethyl ether three times.

Step c: Boc removalfrom MIE 4

The Boc protecting group was removed from MMAE 4 in the presence of TFA (Scheme 5).

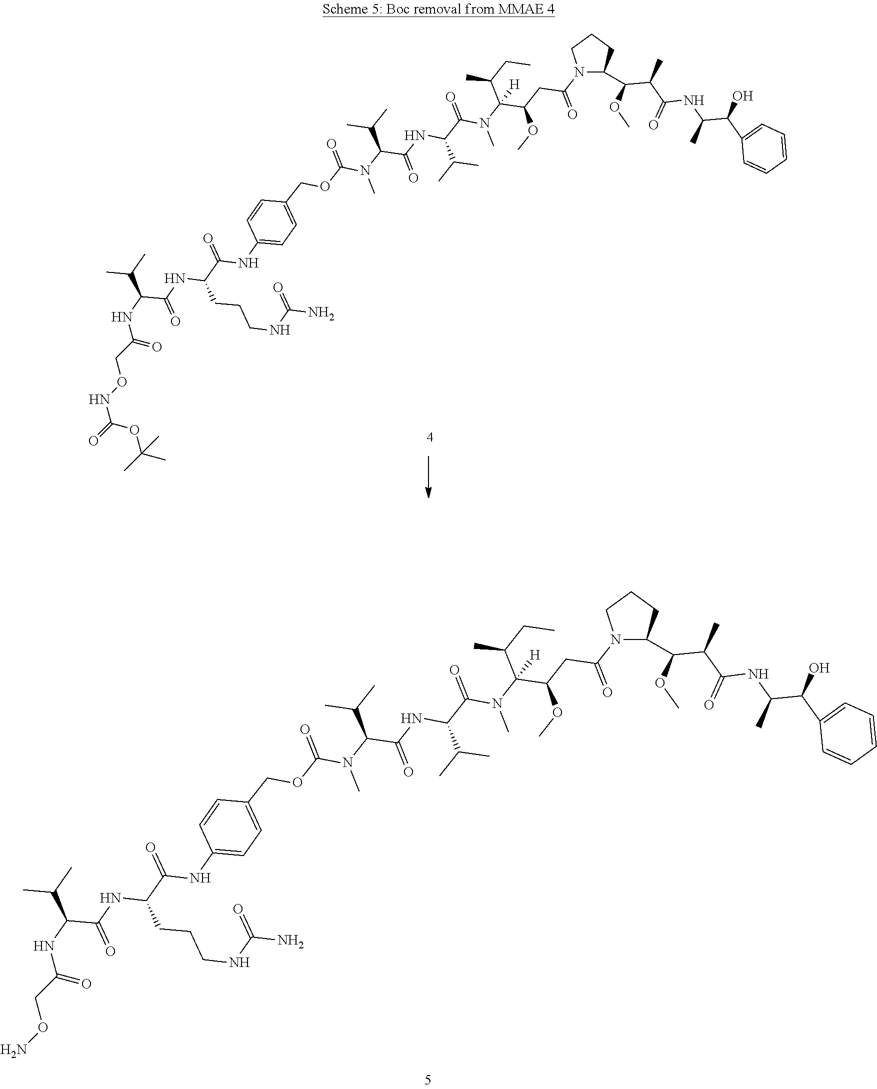 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)CONC(=O)OC(C)(C)C)C(C)C)cc1)C(C)C ; CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)CON)C(C)C)cc1)C(C)C]

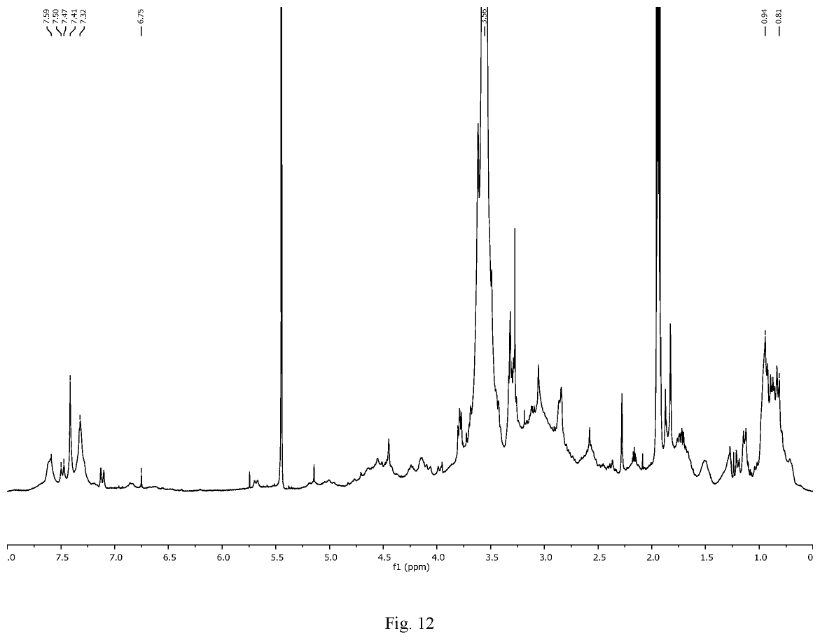

45 mg of MMAE 4 were placed in a RBF and 3 mL of DCM/TFA/TIS (82.5:15:2.5 v/v %) were then added. The RBF was placed in an ice bath and the reaction was monitored by TLC. After 2.5 hours, the reaction solvents were evaporated under vacuum. MMAE 5 was washed with diethyl ether once and then dissolved in DMA (400 μL) for oxime coupling. Samples were taken for LC-MS and HPLC analyses. LC-MS confirmed the desired product MMAE 5 was obtained as a peak with m/z=598.95 was observed (see FIG. 11).

### Example 3: MMAE Conjugation to the Polyamides

MMAE 5 was conjugated to polyamide 2 via oxime bond formation (Scheme 6).

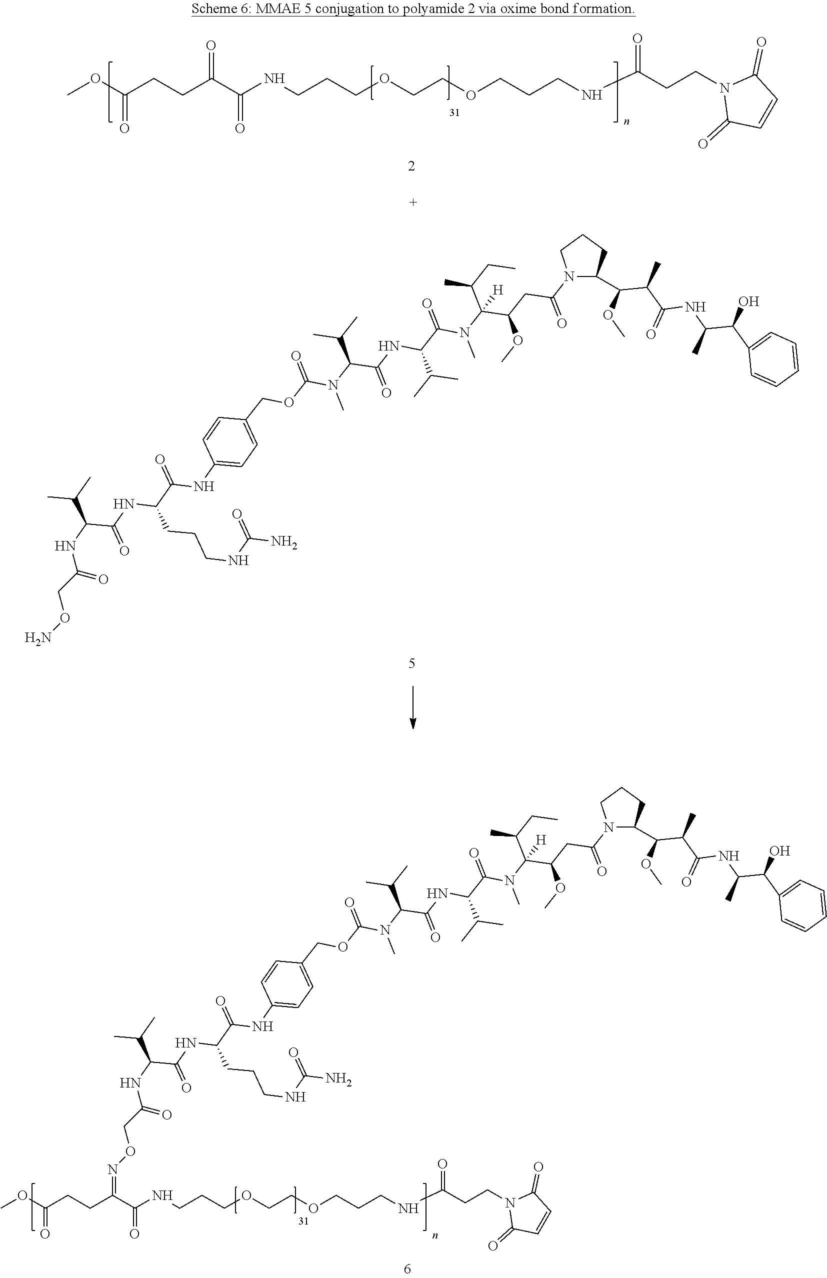

30 mg of polyamide 2 were left stirring at RT in 1 mL DMA until completely dissolved. The solution was then transferred to a reaction vessel containing an estimated 20 mg of MMAE 5 in 200 μL DMA. An additional 600 μL DMA were added. Finally, 200 μL H₂O were added to the RBF. The reaction was left stirring at RT for 24 hours. 20 μL of acetone were then added to quench the reaction and shift the MMAE peak to longer retention times for RP-HPLC purification. 50 mg of MMAE polyamide conjugate 6 in 2 mL of DMA/H₂O (90:10 v/v %) were purified by semi-preparative RP-HPLC (C18 column).

¹H NMR signals of MMAE polyamide conjugate 6 after purification by RP-HPLC is given in FIG. 12. Further analysis is required to confirm the signal assignments and provisional assignments are described as follows: ¹H NMR showed signals characterizing the presence of PEG groups at δ=3.56 ppm, maleimide group at δ=6.75 ppm and MMAE aliphatic (δ=0.94-0.81 ppm) and aromatic (δ=7.59-7.32 ppm) groups.

### Example 4: ADC Preparation by Conjugation of MMAE Polyamide Conjugate 6 to Trastuzumab

MMAE polyamide Trastuzumab ADC was generated by the conjugation of MMAE polyamide conjugate 6 containing maleimide group to the thiol groups of the cysteine residues, following reduction of the disulfide bonds, via the formation of thioether bonds. Conjugation reactions were set up using partially reduced Herceptin (Trastuzumab) and non-reduced Herceptin as described in Table 1.

TABLE 1
Conditions used for the conjugation of MMAE polyamide conjugate 6 to
Trastuzumab (Tris(2-carboxyethyl) phosphine, TCEP)

| | | Reduction | Conjugation |
|---|---|---|---|
| | | conditions | conditions (MMAE |
| | | (TCEP molar | polyamide conjugate 6 molar |
| | | excess, | excess, temperature, time, |
| Antibody | Sample | temperature, time) | DMA) |
| Herceptin | 1 | 3x, 25° C., 2 h | 10x, 30° C., 2 h, 12% |
| | 2 | 25° C., 2 h | 10x, 30° C., 2 h, 12% |

An IgG purification kit utilising protein A coupled bead columns was used to remove excess of conjugate 6 leaving only MMAE polyamide Trastuzumab ADC and uncoupled Trastuzumab.

### Example 5: Cell Viability Assay on the MMAE Polyamide Trastuzumab ADC

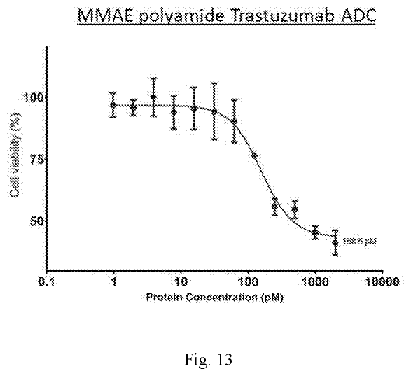

MMAE polyamide Trastuzumab ADC was tested for activity against SKBR3 cells (breast cancer cell line) (see FIG. 13). The sample was added to cells for 72 h before application of the CellTitre Glo luminescence reagent. Concentration is given as pM of protein.

MMAE polyamide Trastuzumab ADC is active on SKBR3 cells with an estimated protein concentration IC₅₀ of 158.5 pM.

1. An antibody-drug conjugate comprising:
(i) an antibody or antigen-binding fragment thereof,
(ii) a polymer comprising a repeat unit of Formula (I′): 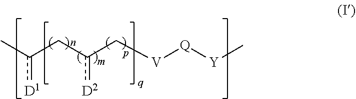
wherein:
each n and each p is independently 0 or an integer between 1 and 6;
each m is independently 0 or an integer between 1 and 4, and preferably at least one m is 1;
V is 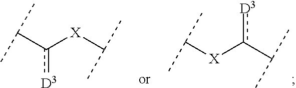
------- is a bond which may be absent or present;
each D¹ is independently O or L¹-B¹;
each D² is independently O or L²-B²;
each D³ is independently O or L³-B³;
wherein L¹ is a linker group or a bond, L² is a linker group or a bond, L³ is a linker group or a bond, and each B¹, B² and B³ is a biologically active moiety;
provided that at least one D¹, D² or D³ group within the polymer is not O, and further provided that when D¹, D² or D³ is O, there is a double bond between the O atom and the carbon atom to which it is attached;
each q is an integer between 1 and 8;
X and Y are independently selected from O, NH, NR′ and S;
R′ is C₁₋₂₀ hydrocarbyl;
Q is selected from —CH₂(NMe(C═O)CH₂)ₒ—, -T¹O(CH₂CH₂O)ₛT²- and -T¹O(CH₂CH₂CH₂O)ₛT²-, wherein T¹ is selected from a divalent methylene, ethylene, propylene or butylene radical, and T² is selected from a divalent methylene, ethylene, propylene or butylene radical;
o is an integer from 0 to 100; and
s is an integer from 0 to 150; and
(iii) a polymer-antibody linker which is covalently bonded to both the antibody and the polymer.

2. An antibody-drug conjugate according to claim 1, wherein the polymer comprises a repeat unit of Formula (I): 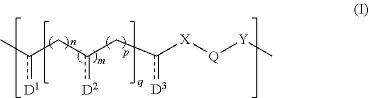
wherein the variables X, Y, D¹, D², D³, n, m and p are as set out above, and Q is selected from -T¹O(CH₂CH₂O)ₛT²- and -T¹O(CH₂CH₂CH₂O)ₛT²-.

3. An antibody-drug conjugate according to claim 1, wherein the polymer-antibody linker is covalently bound to the polymer through the carbon atom of the -CD¹- moiety in Formula (I′) or the Y group in Formula (I′).

4. An antibody-drug conjugate according to claim 1, wherein each D¹ and each D³ is O.

5. An antibody-drug conjugate according to claim 1, wherein (a) each q is 1, and/or (b) each m is 1 or 2, and/or (c) each n is 1, 2 or 3, and/or (d) each p is 0, 1 or 2.

6-8. (canceled)

9. An antibody-drug conjugate according to claim 1, wherein the repeat unit of Formula (I′) has a structure selected from

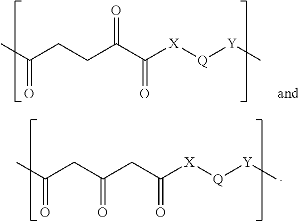

10. An antibody-drug conjugate according to claim 1, wherein the polymer-antibody linker is derived from maleimide, monobromomaleimide, vinyl sulfones, bis(sulfone)s, allenamides, dehydroalanine, alkenes, perfluoroaromatic species, sulfone reagents that are Julia-Kocienski like, N-hydroxysuccinamide-ester activated carboxylate species and ketones.

11. An antibody-drug conjugate according to claim 1 having Formula (III) or (IV): 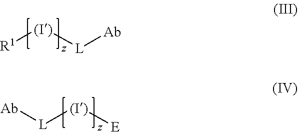
wherein:
(I′) is a repeat unit of the Formula (I′), as defined in claim 1;
Ab is an antibody or antigen-binding fragment thereof,
L is a polymer-antibody linker as defined in claim 1;
R¹ is selected from OH, OR′, SH, SR′, NH₂, NHR′ and NR′₂;
E is selected from H and R′;
R′ is as defined in claim 1; and
z is an integer from 2 to 50.

12. An antibody-drug conjugate according to claim 1, wherein X is O or NH and Y is O or NH, preferably wherein both X and Y are O or wherein both X and Y are NH.

13. An antibody-drug conjugate according to claim 1, wherein Q is —CH₂CH₂O(CH₂CH₂O)ₛCH₂CH₂— or —CH₂CH₂CH₂O(CH₂CH₂O)ₛCH₂CH₂CH₂—, preferably wherein s is from 1 to 100.

14. An antibody-drug conjugate according to claim 1, wherein X-Q-Y is derived from PEG 400, PEG 500, PEG 600, PEG 1000, PEG 1500, PEG 2000 or (poly(ethylene glycol) bis(3-aminopropyl) terminated) 1500.

15. An antibody-drug conjugate according to claim 1, wherein V is —X—(C═O)—, Q is —CH₂(NMe(C═O)CH₂)ₒ—, and Y is —NMe-, wherein the moiety Q is directly bonded to the carbonyl group of moiety V, preferably wherein o is from 5 to 25.

16. An antibody-drug conjugate according to claim 1, wherein each biologically active moiety is the same or different and is a moiety B¹, B² and/or B³, wherein H—B¹, H—B² and/or H—B³ are each independently selected from small molecule drugs, peptides, proteins, peptide mimetics, antibodies, antigens, DNA, mRNA, small interfering RNA, small hairpin RNA, microRNA, PNA, foldamers, carbohydrates, carbohydrate derivatives, non-Lipinski molecules, synthetic peptides and synthetic oligonucleotides, preferably small molecule drugs, preferably wherein H—B¹, H—B² and/or H—B³ comprise at least one hydrazine group, at least one hydrazide group, at least one amine group, at least one aminooxy group, at least one hydroxyl group, at least one thiol group, or at least one carboxylic acid group.

17. An antibody-drug conjugate according to claim 1, wherein at least one, preferably all, of the repeat units of Formula (I′) are selected from:
(a) a repeat unit of Formula (Ia): 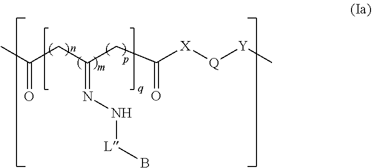
wherein L″ is a bond or —Z¹-L′-Z²—, and B is either the biologically active moiety B², or, if L″ is a bond, B is defined such that B—NH—N is the biologically active moiety B²;
(b) a repeat unit of Formula (Ib) 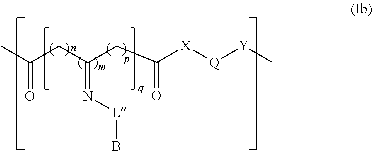
wherein L″ is a bond or —Z¹-L′-Z²—, and B is either the biologically active moiety B², or, if L″ is a bond, B is defined such that B—N is the biologically active moiety B²;
(c) a repeat unit of Formula (Ici) or (Icii): 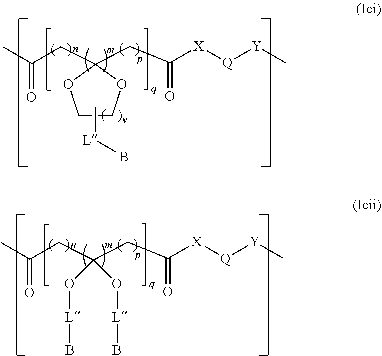
wherein v is an integer from 0 to 4, L″ is a bond or —Z¹-L′-Z²—, and B is either the biologically active moiety B², or, if L″ is a bond, B is defined such that B—O or O—B—O is the biologically active moiety B²;
(d) a repeat unit of Formula (Idi) or (Idii): 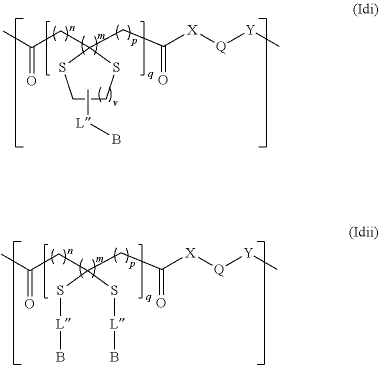
wherein v is an integer from 0 to 4, L″ is a bond or —Z¹-L′-Z²—, and B is either the biologically active moiety B², or, if L″ is a bond, B is defined such that B—S or S—B—S is the biologically active moiety B²; and
(e) a repeat unit of Formula (Ie): 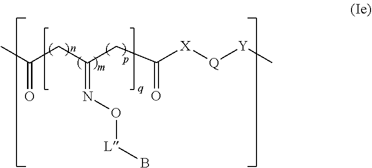
wherein L″ is a bond or —Z¹-L′-Z²—, and B is either the biologically active moiety B², or, if L″ is a bond, B is defined such that B—O—N is the biologically active moiety B²;
L′ is selected from a bond, C₁₋₂₀ alkylene, C₁₋₂₀ alkenylene, C₁₋₂₀ alkynylene, C₆₋₁₀ arylene (e.g. phenylene or naphthylene), C₇₋₂₀ aralkylene, C₃₋₁₀ cycloalkylene, C₄₋₈ heterocycloalkylene, C₅₋₁₀ heteroarylene, C₆₋₂₀ heteroaralkylene, —(O—K)ᵢ—,
—(NH—K)ᵢ—, —(NR′—K)ᵢ—, a polyester having a molecular weight of from 116 to 2000 Da, a polyamide having a molecular weight of from 114 to 2000 Da, and a moiety —W— wherein H—W—OH is an amino acid or a peptide containing from two to twenty naturally-occurring or synthetic amino acid subunits;
Z¹ is selected from —Z—(C═O)—, —Z—O(C═O)—, —Z—NH(C═O)—, —Z—NR′(C═O)—, —Z—S(C═O)—, —Z—(C═NH)—, —Z—O(C═NH)—, —Z—NH(C═NH)—, —Z—NR′(C═NH)—, —Z—S(C═NH)— and —Z—(C═NR′)—K—(O—K)ᵢ—, —K—(NH—K)ᵢ—, —K—(NR′—K)ᵢ—, —K(C═O)—(O—K—(C═O))ᵢ—, —K(C═O)—(NH—K—(C═O))ᵢ—K(C═O)—(NR′—K—(C═O))ᵢ—, and a moiety —P— wherein H₂N—P—OH is an amino acid or a peptide containing from two to twenty naturally-occurring or synthetic amino acid subunits;
Z² is selected from a bond, —OZ—, —NHZ—, —NR′Z—, —SZ—, —S—, —ZS—, —OZS—, —NHZS—, —NR′ZS—, —SZS—, —Z—(C═O)—, —Z—O(C═O)—, —Z—NH(C═O)—, —Z—NR′(C═O)—, —Z—S(C═O)—, —Z—(C═NH)—, —Z—O(C═NH)—, —Z—NH(C═NH)—, —Z—NR′(C═NH)—, —Z—S(C═NH)—, —Z—(C═NR′)—, —Z—O(C═NR)—, —Z—NH(C═NR′)—, —Z—NR′(C═NR′)—, —Z—S(C═NR′)—, —OZ—(C═O)—, —OZ—O(C═O)—, —OZ—NH(C═O)—, —OZ—NR′(C═O)—, —OZ—S(C═O)—, —OZ—(C═NH)—, —OZ—O(C═NH)—, —OZ—NH(C═NH)—, —OZ—NR′(C═NH)—, —OZ—S(C═NH)—, —OZ—(C═NR′)—, —OZ—O(C═NR)—, —OZ—NH(C═NR)—, —OZ—NR′(C═NR′)—, —OZ—S(C═NR′)—, —NHZ—(C═O)—, —NHZ—O(C═O)—, —NHZ—NH(C═O)—, —NHZ—NR′(C═O)—, —NHZ—S(C═O)—, —NHZ—(C═NH)—, —NHZ—O(C═NH)—, —NHZ—NH(C═NH)—, —NHZ—NR′(C═NH)—, —NHZ—S(C═NH)—, —NHZ—(C═NR′)—, —NHZ—O(C═NR)—, —NHZ—NH(C═NR′)—, —NHZ—NR′(C═NR′)—, —NHZ—S(C═NR′)—, —NR′Z—(C═O)—, —NR′Z—O(C═O)—, —NR′Z—NH(C═O)—, —NR′Z—NR′(C═O)—, —NR′Z—S(C═O)—, —NR′Z—(C═NH)—NR′Z—O(C═NH)—, —NR′Z—NH(C═NH)—, —NR′Z—NR′(C═NH)—, —NR′Z—S(C═NH)—, —NR′Z—(C═NR′)—, —NR′Z—O(C═NR′)—, —NR′Z—NH(C═NR′)—, —NR′Z—NR′(C═NR′)—, —NR′Z—S(C═NR′)—, —SZ—(C═O)—, —SZ—O(C═O)—, —SZ—NH(C═O)—, —SZ—NR′(C═O)—, —SZ—S(C═O)—, —SZ—(C═NH)—, —SZ—O(C═NH)—, —SZ—NH(C═NH)—, —SZ—NR′(C═NH)—, —SZ—S(C═NH)—, —SZ—(C═NR′)—, —SZ—O(C═NR′)—, —SZ—NH(C═NR′)—, —SZ—NR′(C═NR′)—, —SZ—S(C═NR)—, -J-O(C═O)—, —O-J-O(C═O)—, —S-J-O(C═O)—, —NH-J-O(C═O)—, —NR′-J-O(C═O)—, a polyether (e.g. poly(alkylene glycol) having a molecular weight of from 76 to 2000 Da), a polyamine having a molecular weight of from 75 to 2000 Da, a polyester having a molecular weight of from 116 to 2000 Da, a polyamide having a molecular weight of from 114 to 2000 Da, and a moiety —W— wherein H—W—OH is an amino acid or a peptide containing from two to twenty naturally-occurring or synthetic amino acid subunits;
Z is selected from C₁₋₂₀ alkylene, C₁₋₂₀ alkenylene, C₁₋₂₀ alkynylene, C₆₋₁₀ arylene (e.g. phenylene or naphthylene), C₇₋₂₀ aralkylene, C₃₋₁₀ cycloalkylene, C₄₋₈ heterocycloalkylene, C₅₋₁₀ heteroarylene, and C₆₋₂₀ heteroaralkylene;
J is a phenyl group which carries a sugar substituent and, para or ortho to the sugar substituent, a methylene group or a moiety —(CH═CH)ₖ—CH₂—, wherein k is an integer from 1 to 10, further wherein the methylene group or moiety —(CH═CH)ₖ—CH₂— is directly bonded to the —O(C═O)— group proximal to the biologically active moiety B¹, B² or B³, and a carbon of the phenyl ring is directly bonded to the remainder of the linker group distal to the biologically active moiety B¹, B² or B³;
each K is the same or different and represents C₁₋₁₀ alkylene;
i is an integer from 1 to 100, preferably from 1 to 50, and more preferably from 2 to 20; and
R′ is C₁₋₂₀ hydrocarbyl.

18. A pharmaceutical composition comprising an antibody-drug conjugate according to claim 1 and a pharmaceutically acceptable excipient.

19-20. (canceled)

21. A method of treating a disease or condition in a human patient, wherein said method comprises administration of at least one antibody-drug conjugate according to claim 1 to a patient in need thereof, and wherein said disease or condition is selected from inflammatory diseases (e.g. inflammatory bowel disease, rheumatoid arthritis and artherosclerosis), metabolic disorders (e.g. diabetes, insulin resistance, obesity), cancer, bacterial infections (e.g. tuberculosis, pneumonia, endocarditis, septicaemia, salmonellosis, typhoid fever, cystic fibrosis, chronic obstructive pulmonary diseases), viral infections, cardiovascular diseases, neurodegenerative diseases, neurological disorders, behavioral and mental disorders, blood diseases, chromosome disorders, congenital and genetic diseases, connective tissue diseases, digestive diseases, ear, nose, and throat diseases, endocrine diseases, environmental diseases, eye diseases, female reproductive diseases, fungal infections, heart diseases, hereditary cancer syndromes, immune system diseases, kidney and urinary diseases, lung diseases, male reproductive diseases, mouth diseases, musculoskeletal diseases, myelodysplastic syndromes, nervous system diseases, newborn screening, nutritional diseases, parasitic diseases, rare cancers and skin diseases.

22. (canceled)

23. An antibody-drug conjugate according to claim 1 wherein the release of said biologically active moiety from the polymer is pH sensitive and is dependent upon the nature of the bond between said biologically active moiety and the repeat unit of the polymer or the linker group to which it is covalently bound.

24. A targeting agent-drug conjugate comprising:
(i) a targeting agent;
(ii) a polymer comprising a repeat unit of Formula (I′): 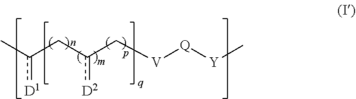
wherein:
each n and each p is independently 0 or an integer between 1 and 6;
each m is independently 0 or an integer between 1 and 4, and preferably at least one m is 1;
V is 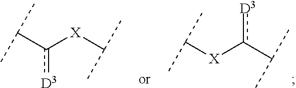
------- is a bond which may be absent or present;
each D¹ is independently O or L¹-B¹;
each D² is independently O or L²-B²;
each D³ is independently O or L³-B³;
L¹ is a linker group or a bond, L² is a linker group or a bond, L³ is a linker group or a bond, and each B¹, B² and B³ is a biologically active moiety;
provided that at least one D¹, D² or D³ group within the polymer is not O, and further provided that when D¹, D² or D³ is O, there is a double bond between the O atom and the carbon atom to which it is attached;
each q is an integer between 1 and 8;
X and Y are independently selected from O, NH, NR′ and S;
R′ is C₁₋₂₀ hydrocarbyl;
Q is selected from —CH₂(NMe(C═O)CH₂)ₒ—, -T¹O(CH₂CH₂O)ₛT²- and -T¹O(CH₂CH₂CH₂O)ₛT²-, wherein T¹ is selected from a divalent methylene, ethylene, propylene or butylene radical, and T² is selected from a divalent methylene, ethylene, propylene or butylene radical;
o is an integer from 0 to 100; and
s is an integer from 0 to 150; and
(iii) a polymer-targeting agent linker which is covalently bonded to both the targeting agent and the polymer.

25. A targeting agent-drug conjugate according to claim 24, wherein the targeting agent is selected from a peptide, a protein, a peptide mimetic, an antibody, an antigen, DNA, mRNA, small interfering RNA, small hairpin RNA, microRNA, PNA, a foldamer, a carbohydrate, a carbohydrate derivative, a non-Lipinski molecule, a synthetic peptide and a synthetic oligonucleotide.
